(19) United States
(12) Patent Application Publication
(10) Pub. No.: US 2002/0077345 A1
(43) Pub. Date: Jun. 20, 2002

(54) Cis-substituted aminocycloalkylpyrrolidine derivatives

(75) Inventors: Makoto Takemura, Tokyo (JP); Hisashi Takahashi, Tokyo (JP); Kazuyuki Sugita, Tokyo (JP); Hitoshi Ohki, Tokyo (JP); Satoru Miyauchi, Tokyo (JP); Rie Miyauchi, Tokyo (JP)

Correspondence Address: SUGHRUE, MION, MACPEAK & SEAS, PLLC, 2100 Pennsylvania Avenue, N.W., Washington, DC, 20037, US

(73) Assignee: DAIICHI PHARMACEUTICAL CO., LTD.

(21) Appl. No.: 09/985,256

(22) Filed: Nov. 2, 2001

Related U.S. Application Data
(63) Continuation of application No. 09/424,112, filed on Nov. 19, 1999.
Application No. 09/424,112 is a 371 of international application No. PCT/JP98/02219, filed on May 20, 1998.

(30) Foreign Application Priority Data
May 21, 1997 (JP) P.HEI-9-131413
May 29, 1997 (JP) P.HEI-9-140643

(51) Int. Cl. A61K 31/40; C07D 207/00

(52) U.S. Cl. 514/408; 548/400

(57) ABSTRACT

Antimicrobial agents being excellent in antimicrobial activity and safety.

Compounds represented by formula (I), its. salts and hydrates thereof:

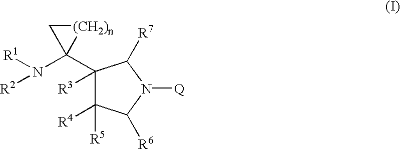

wherein R¹ represents a hydrogen atom or an alkyl group; R² represents a hydrogen atom or an alkyl group; R³ and R⁵, each represents a hydrogen atom; R⁴ represents a hydroxyl group, a halogen atom, a carbamoyl group, an alkyl group, an alkoxyl group or an alkylthio group; R⁶ and R⁷, each independently represents a hydrogen atom or an alkyl group; n is an integer of from 1 to 3; R⁴ and the substituent on the pyrrolidine ring of the following formula:

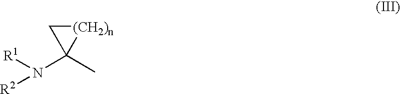

are located in the cis-configuration; and Q represents a partial structure represented by the following formula:

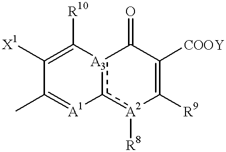

### TECHNICAL FIELD

This invention relates to antimicrobial compounds which are useful as drugs, veterinary drugs, fishery drugs marine drugs and antimicrobial preservatives and antimicrobial agents and antimicrobial preparations containing these compounds.

### BACKGROUND ART

Since norfloxacin was found out, attempts have been made to improve the antimicrobial activities and pharmacokinetics of synthetic quinolone antimicrobial agents. As a result, a number of compounds are clinically available today as chemotherapeutic drugs efficacious against systemic infectious diseases.

In recent years, however, bacteria hyposensitive to these drugs have been increasing in the field of clinical medicine. Moreover, some bacteria tolerant to drugs other than these synthetic quinolone antimicrobial agents become hyposensitive thereto, for example, Staphylococcus aureus (MRSA) insensitive to β-lactam antibiotics. Therefore, it has been urgently required to develop highly efficacious drugs in the field of clinical medicine.

In addition, it is reported that some antimicrobial agents sometimes induce convulsion when administered together with nonsteroidal anti-inflammatory drugs or they have some side effects such as phototoxicity. Thus, it is needed to develop quinolones with elevated safety.

### DISCLOSURE OF INVENTION

Under these circumstances, the present inventors have conducted extensive studies to provide compounds satisfying the above-mentioned requirements. As a result, they have successfully found out that cis-substituted-substituted aminocycloalkylpyrrolidine derivatives represented by the following formula (I), their salts and hydrates thereof have broad antibacterial spectra, in particular, potent antimicrobial activities on quinolone-tolerant bacteria including gram positive bacteria, in particular, MRSA and, at the same time, favorable pharmacokinetics and high safety, thus completing the present invention.

Accordingly, the present invention relates to compounds represented by the following formula (I), its salts and hydrates thereof:

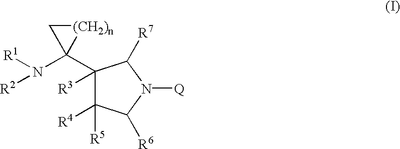

wherein R¹ represents a hydrogen atom or an alkyl group having 1 to 6 carbon atoms;

R² represents a hydrogen atom or an alkyl group having 1 to 6 carbon atoms, provided that the alkyl group may have one or more substituents selected from the group consisting of a hydroxyl group, a halogen atoms and an alkoxyl group having 1 to 6 carbon atoms;

R³ and R⁵ represent each a hydrogen atom;

R⁴ represents a hydroxyl group, a halogen atom, a carbamoyl group, an alkyl group having 1 to 6 carbon atoms, an alkoxyl group having 1 to 6 carbon atoms or an alkylthio group having 1 to 6 carbon atoms, provided that the alkyl group may have one or more substituents selected from the group consisting of a hydroxyl group, a halogen atom and an alkoxyl group having 1 to 6 carbon atoms; and

R⁴ and the substituent on the pyrrolidine ring of the following formula:

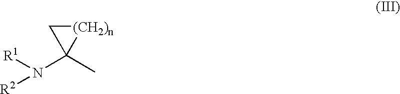

are located in the cis-configuration;

R⁶ and R⁷, each independently represents a hydrogen atom or an alkyl group having 1 to 6 carbon atoms;

n is an integer of from 1 to 3; and

Q represents a partial structure represented by the following formula:

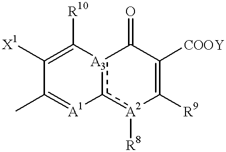

wherein R⁸ represents an alkyl group having 1 to 6 carbon atoms, an alkenyl group having 2 to 6 carbon atoms, a halogenoalkyl group having 1 to 6 carbon atoms, a substituted or unsubstituted cyclic alkyl group having 3 to 6 carbon atoms; a substituted or unsubstituted aryl group, a substituted or unsubstituted heteroaryl group, an alkoxyl group having 1 to 6 carbon atoms or an alkylamino group having 1 to 6 carbon atoms;

R⁹ represents a hydrogen atom or an alkylthio group having 1 to 6 carbon atoms;

R⁹ and R⁸ may form together with a part of the mother nucleus a cyclic structure optionally containing a sulfur atom as a constituting atom thereof and optionally having an alkyl group having 1 to 6 carbon atoms as a substituent;

R¹⁰ represents a hydrogen atom, an amino group, a hydroxyl group, a thiol group, a halogenomethyl group, an alkyl group having 1 to 6 carbon atoms, an alkenyl group having 2 to 6 carbon atoms, an alkynyl group having 2 to 6 carbon atoms or an alkoxyl group having 1 to 6 carbon atoms, provided that the amino group may have one or more substituents selected from the group consisting of a formyl group, an alkyl group having 1 to 6 carbon atoms and an acyl group having 2 to 5 carbon atoms;

X¹ represents a halogen atom or a hydrogen atom;

A¹ represents a nitrogen atom or a partial structure represented by the following formula (II):

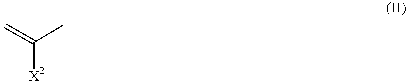

wherein X² represents a hydrogen atom, an amino group, a halogen atom, a cyano group, a halogenomethyl group, a halogenomethoxyl group, an alkyl group having 1 to 6 carbon atoms, an alkenyl group having 2 to 6 carbon atoms, an alkynyl group having 2 to 6 carbon atoms or an alkoxyl group having 1 to 6 carbon atoms, provided that the amino group may have one or more substituents selected from the group consisting of a formyl group, an alkyl group having 1 to 6 carbon atoms and an acyl group having 2 to 5 carbon atoms; and

X² and R⁸ may form together with a part of the mother nucleus a cyclic structure optionally containing an oxygen atom, a nitrogen atom or a sulfur atom as a constituting atom thereof and optionally having an alkyl group having 1 to 6 carbon atoms as a substituent;

A² and A³, each represents a nitrogen atom or a carbon atom, provided that A², A³ and the carbon atom to which they are bonded may form together with the bonds among them, expressed in dotted lines, a partial structure represented by the following formula:

>C═C(A¹═)—N(R⁸)— or >N—C(A¹═)═C(R⁸)—; and

Y represents a hydrogen atom, a phenyl group, an acetoxymethyl group, a pivaloyloxymethyl group, an ethoxycarbonyl group, a choline group, a dimethylaminoethyl group, a 5-indanyl group, a phthalidinyl group, a 5-alkyl-2-oxo-1,3-dioxol-4-ylmethyl group, a 3-acetoxy-2-oxobutyl group, an alkyl group having 1 to 6 carbon atoms, an alkoxymethyl group having 2 to 7 carbon atoms or a phenylalkyl group composed of an alkyl group having 1 to 6 carbon atoms and a phenyl group.

The present invention further provides:

the compounds of the formula (I), wherein Q has a structure represented by the following formula, its salts and hydrates thereof:

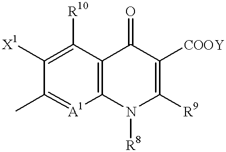

or the following formula:

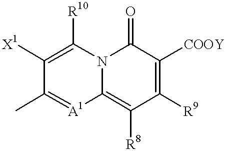

wherein R⁸, R⁹, R¹⁰, A¹, X¹ and Y are each as defined above;

the compounds of the formula (I), wherein Q has a structure represented by the following formula, its salts and hydrates thereof:

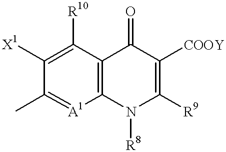

wherein R⁸, R⁹, R¹⁰ A¹, X¹ and Y are each as defined above;

the compounds of the formula (I), wherein Q is a 6-carboxy-9-fluoro-2,3-dihydro-3-(S)-methyl-7-oxo-7H-pyrido[1,2,3-de][1,4]benzoxazin-10-yl group (following formula), its salts and hydrates thereof:

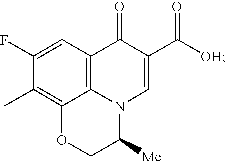

the compound of the formula (I), wherein Q is an 8-amino-6-carboxy-9-fluoro-2,3-dihydro-3-(S)-methyl-7-oxo-7H-pyrido[,1,2,3-de][1,4]benzoxazin-10-yl group (following formula), its salts and hydrates thereof:

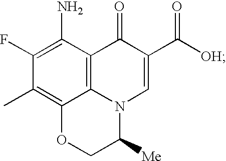

the compounds of the formula (I), wherein Q is a 5-amino-3-carboxy-6-fluoro-1-[2-(S)-fluoro-1-(R)-cyclopropyl]-1,4-dihydro-8-methoxy-4-oxoquinolin-7-yl group (following formula), its salts and hydrates thereof:

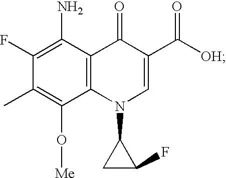

the compounds of the formula (I), wherein Q is a 5-amino-3-carboxy-6-fluoro-1-[2-(S)-fluoro-1-(R)-cyclopropyl]-1,4-dihydro-8-methyl-4-oxoquinolin-7-yl group (following formula), its salts and hydrates thereof:

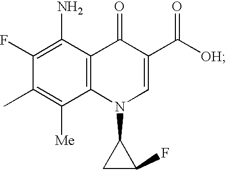

the compounds of the formula (I), wherein Q is a 3-carboxy-6-fluoro-1-[2-(S)-fluoro-1-(R)-cyclopropyl]-1,4-dihydro-8-methoxy-4-oxoquinolin-7-yl group (following formula), its salts and hydrates thereof:

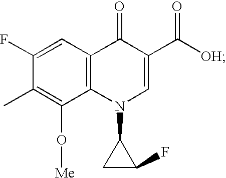

the compound of the formula (I), wherein the substituent R⁴ is a halogen atom, its salts and hydrates thereof;

the compounds of the formula (I), wherein the substituent R⁴ is a fluorine atom, its salts and hydrates thereof;

the compounds of the formula (I), wherein n is 1 or 2, its salts and hydrates thereof;

the compounds of the formula (I), wherein n is 1, its salts and hydrates thereof;

the compounds of the formula (I), wherein the substituent R⁴ is a fluorine atom and n is 1 or 2, its salts and hydrates thereof;

the compounds of the formula (I), wherein the substituent R⁴ is a fluorine atom and n is 1, its salts and hydrates thereof;

the compounds of the formula (I), wherein the substituent R⁸ is a halogenocyclopropyl group, its salts and hydrates thereof;

the compounds of the formula (I), wherein the substituent R⁸ is a 1,2-cis-2-halogenocyclopropyl group, its salts and hydrates thereof;

the compounds of the formula (I), wherein the substituent R⁸ is a stereochemically pure one, its salts and hydrates thereof;

the compounds of the formula (I), wherein the substituent R⁸ is a (1R,2S)-2-halogenocyclopropyl group, its salts and hydrates thereof;

the compounds of the formula (I), wherein the substituent R⁸ is a (1R,2S)-2-fluorocyclopropyl group, its salts and hydrates thereof;

the compounds represented by the formula (I), which are stereochemically pure ones, its salts and hydrates thereof;

the compounds of the formula (I), wherein the substituent X¹ is a halogen atom its salts and hydrates thereof;

the compounds of the formula (I), wherein X¹ is a fluorine atom, its salts and hydrates thereof;

10-[4-(R)-(1-aminocyclopropyl)-3-(S)-fluoro-1-pyrrolidinyl]-9-fluoro-2,3-dihydro-3-(S)-methyl-7-oxo-7H-pyrido[1,2,3-de][1,4]benzoxazine-6-carboxylic acid, its salts and hydrates thereof;

8-amino-10-[4-(R)-(1-aminocyclopropyl)-3-(S)-fluoro-1-pyrrolidinyl]-9-fluoro-2,3-dihydro-3-(S)-methyl-7-oxo-7H-pyrido[1,2,3-de][1,4]benzoxazine-6-carboxylic acid, its salts and hydrates thereof;

5-amino-7-[4-(R)-(1-aminocyclopropyl)-3-(S)-fluoro-1-pyrrolidinyl]-6-fluoro-1-[2-(S)-fluoro-1-(R)-cyclopropyl]-1,4-dihydro-8-methyl-4-oxoquinoline-3-carboxylic acid, its salts and hydrates thereof;

5-amino-7-[4-(R)-(1-aminocyclopropyl)-3-(S)-fluoro-1-pyrrolidinyl]-6-fluoro-1-[2-(S)-fluoro-1-(R)-cyclopropyl]-1,4-dihydro-8-methoxy-4-oxoquinoline-3-carboxylic acid, its salts and hydrates thereof;

7-[4-(R)-(1-aminocyclopropyl)-3-(S)-fluoro-1-pyrrolidinyl]-6-fluoro-1-[2-(S)-fluoro-1-(R)-cyclopropyl]-1,4-dihydro-8-methoxy-4-oxoquinoline-3-carboxylic acid, its salts and hydrates thereof;

drugs containing as the active ingredient the above-mentioned compounds, hydrates thereof, salts of the compounds or hydrates of the salts.

antimicrobial agents containing as the active ingredient the above-mentioned compounds, hydrates thereof, salts of the compounds or hydrates of the salts; etc.

The present invention further relates to compounds represented by the following formula, its salts and hydrates thereof:

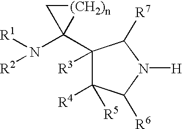

wherein R¹ represents a hydrogen atom or an alkyl group having 1 to 6 carbon atoms;

R² represents a hydrogen atom or an alkyl group having 1 to 6 carbon atoms, provided that the alkyl group may have one or more substituents selected from the group consisting of a hydroxyl group, a halogen atom and an alkoxyl group having 1 to 6 carbon atoms;

R³ and R⁵ represent each a hydrogen atom;

R⁴ represents a hydroxyl group, a halogen atom, a carbamoyl group, an alkyl group having 1 to 6 carbon atoms, an alkoxyl group having 1 to 6 carbon atoms or an alkylthio group having 1 to 6 carbon atoms, provided that the alkyl group may have one or more substituents selected from the group consisting of a hydroxyl group, a halogen atom and an alkoxyl group having 1 to 6 carbon atoms; and

R⁴ and the substituent of the following formula:

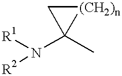

are located in the cis-configuration;

R⁶ and R⁷, each independently represents a hydrogen atom or an alkyl group having 1 to 6 carbon atoms; and

n is an integer of from 1 to 3.

The present invention furthermore relates to the above-mentioned compounds, wherein the substituent R ⁴ is a halogen atom, its salts and hydrates thereof;

the above-mentioned compounds, wherein the substituent R⁴ is a fluorine atom, their salts and hydrates thereof;

the above-mentioned compounds, wherein n is 1 or 2, its salts and hydrates thereof;

the above-mentioned compounds, wherein n is 1, its salts and hydrates thereof;

the above-mentioned compounds, wherein the substituent R⁴ is a fluorine atom and n is 1 or 2, its salts and hydrates thereof;

the above-mentioned compounds, wherein the substituent R⁴ is a fluorine atom and n is 1, its salts and hydrates thereof;

4-(R)-(1-aminocyclopropyl)-3-(S)-fluoro-1-pyrrolidine, its salts and hydrates thereof; etc.

Now, the substituents in the compounds of the present invention represented by the formula (I) will be explained.

The substituent R¹ represents a hydrogen atom or an alkyl group having 1 to 6 carbon atoms. This alkyl group may be either a liner or branched one having 1 to 6 carbon atoms. Preferable examples thereof include methyl, ethyl, n-propyl and isopropyl groups.

R² represents a hydrogen atom or an alkyl group having 1 to 6 carbon atoms. This alkyl group may have one or more substituents selected from the group consisting of a hydroxyl group, a halogen atom, an alkylthio group having 1 to 6 carbon atoms and an alkoxy group having 1 to 6 carbon atoms.

This alkyl group may be either a liner or branched one having 1 to 6 carbon atoms. Preferable examples thereof include methyl, ethyl, n-propyl and isopropyl groups.

When this alkyl group is substituted by a hydroxyl group, the alkyl group may be either a linear or branched one having 1 to 6 carbon atoms and the hydroxyl group is preferably attached to the terminal carbon atom of the alkyl group. Preferable examples of the alkyl group having a hydroxyl group include hydroxymethyl, 2-hydroxyethyl, 2-hydroxypropyl and 3-hydroxypropyl groups.

When this alkyl group is substituted by a halogen atom, the alkyl group may be either a linear or branched one having 1 to 6 carbon atoms and a fluorine atom is preferable as the halogen atom.

When this alkyl group is substituted by an alkylthio group, the alkyl group may be either a linear or branched one having 1 to 6 carbon atoms. Also, the alkylthio group may be either a linear or branched one having 1 to 6 carbon atoms. Examples of the alkyl group having an alkylthio group include alkylthiomethyl, alkykthioethyl and alkylthiopropyl groups. It is preferable that the alkylthio group has up to 3 carbon atoms too. Namely, still preferably examples thereof include methylthiomethyl, ethylthiomethyl and methylthioethyl groups.

When this alkyl group is substituted by an alkoxyl group, the alkyl group may be either a linear or branched one having 1 to 6 carbon atoms. Also, the alkoxyl group may be either a linear or branched one having 1 to 6 carbon atoms. Examples of the alkyl group having an alkoxyl group include alkoxymethyl, alkoxethyl and alkoxypropyl groups. It is preferable that the alkoxyl group has up to 3 carbon atoms too. Namely, still preferably examples thereof include methoxymethyl, ethoxymethyl and methoxyethyl groups.

The substituents R³ and R⁵, each represents a hydrogen atom. These hydrogen atoms are located in the cis-configuration with regard to the pyrrolidine ring.

The substituent R⁴ represents a hydroxyl group, a halogen atom, a carbamoyl group, an alkyl group having 1 to 6 carbon atoms, an alkoxyl group having 1 to 6 carbon atoms or an alkylthio group having 1 to 6 carbon atoms. This alkyl group may have one or more substituents selected from the group consisting of a hydroxyl group, a halogen atom and an alkoxy group having 1 to 6 carbon atoms.

Preferable examples of the halogen atom are fluorine and chlorine atoms.

Although the alkyl group may either a linear or branched one having 1 to 6 carbon atoms, preferable examples thereof include methyl, ethyl, n-propyl and isopropyl groups.

Although the alkoxyl group may either a linear or branched one having 1 to 6 carbon atoms, preferable examples thereof include methoxyl and ethoxyl groups.

Although the alkylthio group may either a linear or branched one having 1 to 6 carbon atoms, preferable examples thereof include methylthio and ethylthio groups.

When this alkyl group is substituted by a hydroxyl group, the alkyl group may be either a linear or branched one having 1 to 6 carbon atoms and the hydroxyl group is preferably attached to the terminal carbon atom of the alkyl group. Preferable examples of the hydroxylated alkyl group having 1 to 6 carbon atoms include hydroxymethyl, 2-hydroxyethyl and 3-hydroxypropyl groups.

When this alkyl group is substituted by a halogen atom, preferable examples of the halogen atom include fluorine and chlorine atoms, and a fluorine atom is still preferable. The alkyl group may be either a linear or branched one having 1 to 6 carbon atoms.

When this alkyl group having 1 to 6 carbon atoms is substituted by an alkoxyl group, each alkyl moiety may be either a linear or branched one having 1 to 6 carbon atoms. Preferable examples thereof include alkoxymethyl or alkoxethyl groups and methoxymethyl, ethoxymethyl and 2-methoxyethyl groups are still preferable therefor.

The substituent R⁴ and the substituent on the pyrrolidine ring of the following formula:

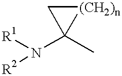

are located in the cis-configuration, which is one of the characteristics of the compounds of the present invention.

The substituents R⁶ and R⁷, each independently represents a hydrogen atom or an alkyl group having 1 to 6 carbon atoms. Although the alkyl group may either a linear or branched one having 1 to 6 carbon atoms, preferable examples thereof include methyl, ethyl, n-propyl and isopropyl groups.

n is an integer of from 1 to 3. Namely, the ring may range from a cyclopropane ring to a cyclopentane ring. In the compounds of the present invention, this moiety has a cyclic structure, which is another characteristics of the present invention. It is particularly preferable that n is 1.

Q is a partial structure of a fused heterocycles represented by the following formula:

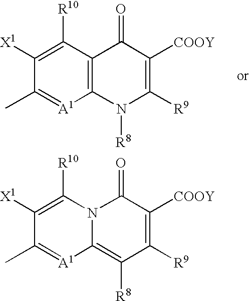

The substituent R⁸ represents an alkyl group having 1 to 6 carbon atoms, an alkenyl group having 2 to 6 carbon atoms, a halogenoalkyl group having 1 to 6 carbon atoms, a substituted or unsubstituted cyclic alkyl group having 3 to 6 carbon atoms; a substituted or unsubstituted aryl group, a substituted or unsubstituted heteroaryl group, an alkoxyl group having 1 to 6 carbon atoms or an alkylamino group having 1 to 6 carbon atoms.

An ethyl group is particularly preferable as the alkyl group having 1 to 6 carbon atoms. A vinyl or 1-isopropenyl group is preferable as the alkenyl group having 2 to 6 carbon atoms. A 2-fluoroethyl group is preferable as the halogenoalkyl group having 1 to 6 carbon atoms. A cyclopropyl and 2-halogenocyclopropyl groups are preferable as the substituted or unsubstituted cyclic alkyl group having 3 to 6 carbon atoms. As the halogen atom in the 2-halogenocyclopropyl group, a fluorine atom is preferable in particular.

Examples of the substituted or unsubstituted aryl group include a phenyl group, etc. optionally having 1 to 3 substituents selected from the group consisting of a halogen atom (e.g., fluorine, chlorine or bromine), a lower alkyl group having 1 to 6 carbon atoms, a hydroxyl group, an amino group, a nitro group, a lower alkoxyl group having 1 to 6 carbon atoms, etc. Preferable examples thereof include phenyl, 2-fluorophenyl, 4-fluorophenyl, 2,4-difluorophenyl and 2-fluoro-4-hydroxyphenyl groups.

A heteroaryl group is a substituent derived from an aromatic heterocyclic compound containing at least one heteroatom selected from among nitrogen, oxygen and sulfur atoms. Examples thereof include pyridyl and pyrimidyl groups. Preferable examples of the substituents on these rings include an alkyl group and a halogen atom. A methoxyl group is preferable as the alkoxyl group having 1 to 6 carbon atoms, while a methylamino group is preferable as the alkylamino group having 1 to 6 carbon atoms.

Preferable examples of the substituent R⁸ include a cyclic alkyl group and a halogenocycloalkyl group. Among these substituents, a cyclopropyl group or a 2-halogenocyclopropyl group is preferable therefor. As the halogen atom, a fluorine atom is preferable.

The substituent R⁹ represents a hydrogen atom or an alkylthio group having 1 to 6 carbon atoms. Alternatively, R⁹ and R⁸ may form together with a part of the mother nucleus (containing A² to which R⁸ is bonded and the carbon atom to which R⁹ is bonded) a cyclic structure. The ring thus formed may contain a sulfur atom as a constituting atom thereof and have an alkyl group having 1 to 6 carbon atoms as a substituent. The ring thus formed is a 4- to 6-membered one which is either saturated, partly saturated or unsaturated.

The substituent X¹ represents a halogen atom or a hydrogen atom. When it is a halogen atom, a fluorine atom is preferable therefor. Among all, a fluorine or hydrogen atom is preferable as this substituent.

The substituent R¹⁰ represents a hydrogen atom, an amino group, a hydroxyl group, a thiol group, a halogenomethyl group, an alkyl group having 1 to 6 carbon atoms, an alkenyl group having 2 to 6 carbon atoms, an alkynyl group having 2 to 6 carbon atoms or an alkoxyl group having 1 to 6 carbon atoms. The amino group may have one or more substituents selected from the group consisting of a formyl group, an alkyl group having 1 to 6 carbon atoms and an acyl group having 2 to 5 carbon atoms.

Although the alkyl group may either a linear or branched one having 1 to 6 carbon atoms, preferable examples thereof include methyl, ethyl, n-propyl and isopropyl groups. The alkenyl group is a linear or branched one having 2 to 6 carbon atoms and a vinyl group is preferable therefor. Although the alkynyl group may either a linear or branched one having 2 to 6 carbon atoms, an ethynyl group is preferable therefor. One to three fluorine atoms are particularly preferable as the halogen in the haolenomethyl group. Although the alkoxyl group may be one having 1 to 6 carbon atoms, a methoxymethyl group is preferable therefor.

Preferable examples of the substituent R¹⁰ include alkyl and amino groups. Among all, a methyl group and an unsubstituted amino group are particularly preferable therefor.

When the substituent R¹⁰ is an amino, a hydroxyl group or a thiol group, it may be protected by protective groups usually employed in the art.

Examples of such protective groups include alkoxycarbonyl groups (e.g., tert-butoxycarbonyl and 2,2, 2-trichloroethoxycarbonyl), aralkyloxycarbonyl groups (e.g., benzyloxcarbonyl, p-methoxybenzyloxycarbonyl and p-nitrobenzyloxycarbonyl), acyl groups (e.g., acetyl, methoxyacetyl, trifluoroacetyl, chloroacetyl, pivaloyl, formyl and benzoyl), alkyl or aralkyl groups (e.g., tert-butyl, benzyl, p-nitrobenzyl, p-methoxybenzyl and triphenylmethyl), ethers (e.g., methoxymethyl, tert-butoxymethyl, tetrahydropyranyl and 2,2, 2-trichloroethoxymethyl) and substituted silyl groups (e.g., trimethylsilyl, isopropyldimethylsilyl, tert-butyldimethylsilyl, tribenzylsily, and tert-butyldiphenylsilyl). Compounds carrying substituents protected by these groups are preferable particularly as intermediates in production processes.

When A¹ represents a partial structure represented by the following formula:

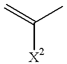

X² represents a hydrogen atom, an amino group, a halogen atom, a cyano group, a halogenomethyl group, a halogenomethoxy group, an alkyl group having 1 to 6 carbon atoms, an alkenyl group having 2 to 6 carbon atoms, an alkynyl group having 2 to 6 carbon atoms or an alkoxyl group having 1 to 6 carbon atoms. The amino group may have one or more substituents selected from the group consisting of a formyl group, an alkyl group having 1 to 6 carbon atoms and an acyl group having 2 to 5 carbon atoms.

Although the alkyl group may be either a linear or branched one having 1 to 6 carbon atoms, methyl and ethyl groups are preferable therefor. Although the alkenyl group may be either a linear or branched one having 2 to 6 carbon atoms, a vinyl group is preferable therefor. Although the alkynyl group may be either a linear or branched one having 2 to 6 carbon atoms, an ethynyl group is preferable therefor. One to three fluorine atoms are particularly preferable as the halogen in the halogenomethyl group. Although the alkoxyl group may be one having 1 to 6 carbon atoms, a methoxyl group is preferable therefor. One to three fluorine atoms are particularly preferable as the halogen in the halogenomethoxyl group.

Among these substituents, an alkyl or alkoxyl group is preferable, and methyl and methoxyl groups are still preferable.

X² and R⁸ may form together with a part of the mother nucleus (containing A² to which R⁸ is bonded and the carbon atoms to which X² is bonded) a cyclic structure which is a 4- to 7-membered ring being either saturated, partly saturated or unsaturated. This ring may contain an oxygen atom, a nitrogen atom or a sulfur atom as a constitutent atom thereof and optionally have an alkyl group having 1 to 6 carbon atoms as a substituent.

As an example of the fused ring system thus formed, 2,3-dihydro-7-oxo-7H-pyrido[1,2,3-de][1,4]benzoxazin-6-carboxylic acid structure is the preferable one and the 3(S)-methyl compound is particularly preferable.

When A¹ is a partial structure represented by the following formula:

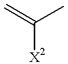

examples of the preferable combination of R¹⁰ with X²include those wherein R¹⁰ is an amino group, a hydrogen atom, a hydroxyl group or an alkyl group having 1 to 6 carbon atoms and X² is an alkyl group having 1 to 6 carbon atoms, an alkoxyl group having 1 to 6 carbon atoms, a halogen atom, a halogenomethoxyl group or a hydrogen atom.

Examples of the still preferable combination thereof are those wherein R¹⁰ is an amino group, a hydrogen atom, a hydroxyl group or a methyl group and X² is a methyl group, a methoxyl group, a fluorine atom, a chlorine atom, a difluoromethoxyl group or a hydrogen atom.

Examples of the particularly preferable combination thereof are those wherein R¹⁰ is an amino group, a hydrogen atom, a hydroxyl group or a methyl group and X² is a methyl group or a methoxyl group.

Preferable examples of R¹⁰ and X² are cited above. On the other hand, a fluorine atom is preferable as X¹.

When the substituents X¹ and X² are each a halogen atom, it is particularly preferable that X¹ is a fluorine atom and X² is a fluorine or chlorine atom.

Next, the halogenocyclopropyl group represented by R⁸ will be explained.

Examples of the halogen atom as the substituent include fluorine and chlorine atoms and a fluorine atom is particularly preferable therefor.

With respect to the stereochemical environment in this moiety, it is particularly preferable that the halogen atom and the pyridonecarboxylate moiety are located at the cis-configuration regarding the cyclopropane ring.

There are so-called enantiomers with respect to the cis-2-halogenocyclopropyl moiety of R⁸. These isomers each shows a potent antimicrobial activity and a high safety.

The compounds of the present invention show excellent characteristics owing to the substituent represented by the following formula (III) located on the pyrrolidine ring:

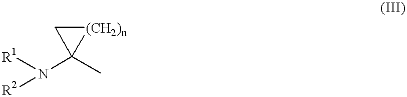

The compounds of the present invention are further characterized in that this substituent and the substituent R⁴ are located in the cis-configuration. (As a matter of course, the substituents R³ and R⁵ are located in the cis-configuration too.) It has been clarified that the compounds of the present invention have excellent characteristics in safety since these substituents are located in the cis-configuration. That is to say, favorable properties such as a decrease in the acute toxicity and negativeness in the micronuclear test are thus confirmed. Especially, it has been clarified that the compounds of the present invention, which are characterized in that the substituent of the formula (III) and the substituent R⁴ are located in the cis-configuration, are superior in a decrease in the acute toxicity as compared with those compounds having the substituent of the formula (III) and the substituent R⁴ in the trans-configuration.

The excellent characteristics in safety of the compounds of the present invention are obvious when the cyclic moiety in the substituent represented by the formula (III) is a 3-membered ring. Also, these characteristics become obvious when the substituent R⁴ is a fluorine atom. As preferable examples of the compounds according to the present invention, namely, those wherein n is 1 and the substituent R⁴ is a fluorine atom are exemplified.

In the compounds represented by the formula (I) of the present invention, the substituent R⁴ and the substituent having a cyclic structure of the formula (III) are located in the cis-configuration. More particularly speaking, the following two isomers occur with respect to this moiety:

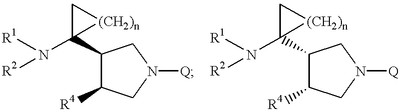

The present inventors consider that the isomer represented by the following formula is preferred to another one:

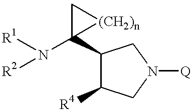

When a compound represented by the formula (I) according to the present invention has a structure allowing the existence of diastereomers, it is preferable that a compound comprised of a single diastereomer is administered to human being or animals. The term “comprised of a single diastereomer” as used herein means not only one being completely free from other diastereomer(s) but also one having a certain degree of chemically purity. That is to say, it may contain other diastereomer(s) so long as neither the physical constants nor the physiological activities thereof are affected thereby.

Also, the term “stereochemically pure” as used herein means a compound consisting of one of isomers, when the compound has two or more isomers due to asymmetric carbon atom(s) contained therein. The term “pure” of this case can be understood in the same manner as the abovementioned case.

The pyridonecarboxylic acid derivatives of the present invention may be in a free state. Alternatively, they may be converted into acid addition salts or carboxylates thereof. Examples of the acid addition salts include inorganic acid salts (e.g., hydrochloride, sulfate, nitrate, hydrobromide, hydroiodide and phosphate) and organic acid salts (e.g., acetate, methanesulfonate, benzenesulfonate, toluenesulfonate, citrate, maleate, fumarate and lactate).

Examples of the carboxylates include alkali metal salts (e.g., lithium salt, sodium salt and potassium salt), alkaline earth metal salts (e.g., magnesium salt and calcium salt), ammonium salt, triethylamine salt, N-methylglucamine salt and tris-(hydroxymethyl) aminomethane salt. Either inorganic salts or organic salts are usable therefor.

These free pyridonecarboxylic acid derivatives or their acid addition salts or carboxylates may occur as hydrates thereof.

On the other hand, quinolone derivatives wherein the carboxylate moiety (—COOY) is an ester are useful as intermediates in the synthesis or prodrugs. For example, alkyl esters, benzyl esters, alkoxyalkyl esters, phenylalkyl esters and phenyl esters are useful as intermediates in the synthesis.

The esters usable as prodrugs are those which are easily cleaved in vivo to thereby form free carboxylates. Examples thereof include acetoxymethyl ester, pivaloyloxymethyl ester, ethoxycarbonyl ester, choline ester, dimethylaminoethyl ester, 5-indanyl ester and oxoalkyl esters such as phthalidinyl ester, 5-alkyl-2-oxo-1,3-dioxol-4-ylmethyl ether and 3-acetoxy-2-oxobutyl. ester and the like.

The compounds represented by the formula (I) of the present invention can be produced by various methods. A preferable example of these method comprises reacting a compound represented by the following formula (IV):

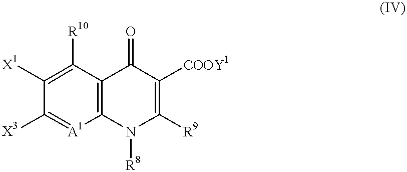

wherein X³ represents a group serving as a leaving group such as a fluorine atom, a chlorine atom, a bromine atom, a substituted or unsubstituted phenylsulfonyl group or a substituted or unsubstituted alkylsulfonyl group having 1 to 3 carbon atoms;

Y¹ means Y as defined in the above formula (I) or a boron-containing group represented by the following formula:

—B(Y¹¹)Y¹²

wherein Y¹¹ and Y¹² represent each a fluorine atom or an alkylcarbonyloxy group having 2 to 4 carbon atoms; and

R⁸, R⁹, R¹⁰, A¹ and X¹ are as defined in the above formula (I);

or a compound represented by the following formula (V):

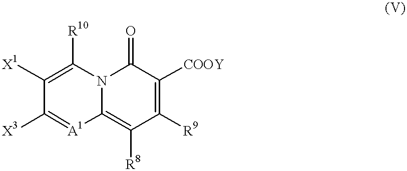

wherein X³ represents a group serving as a leaving group such as a fluorine atom, a chlorine atom, a bromine atom, a substituted or unsubstituted phenylsulfonyl group or a substituted or unsubstituted alkylsulfonyl group having 1 to 3 carbon atoms; and

R⁸, R⁹, R¹⁰, A¹, X¹ and Y are as defined in the above formula (I);

with a compound represented by the following formula (VI) or its acid addition salts:

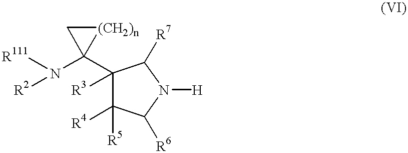

wherein R¹¹¹ has the same meaning as that of R¹ as defined in the above formula (I) or represents a protective group of amino group; and

R², R³, R^{4,} R⁵, R⁶, R⁷ and n are as defined in the above formula (I);

provided that the substituent R⁴ and the substituent containing the cyclic structure bonded to the carbon atom adjacent to the carbon atom to which the substituent R⁴ is bonded are located in the cis-configuration.

Examples of the acid addition salt include inorganic acid salts and organic acid salts. More particularly speaking, organic acid salts (e.g., hydrochloride, sulfate, nitrate, hydrobromide, hydroiodide and phosphate) and organic acid salts (e.g., sulfonates such as methanesulfonate, benzenesulfonate and toluenesulfonate, and carboxylates such as acetate, citrate, maleate, fumarate and lactate) are exemplified.

The reaction may be performed with the use of a solvent or without using any solvent. Any solvent may be employed in the reaction, so long as it remains inert under the reaction conditions. For example, dimethyl sulfoxide, pyridine, acetonitrile, ethanol, chloroform, dimethylformamide, dimethylacetamide, N-methylpyrrolidone, tetrahydrofuran, water, 3-methoxybutanol and mixtures thereof are exemplified as a solvent.

It is preferable that the reaction is performed in the presence of an acid acceptor such as an inorganic base or organic acid base (e.g., alkali metal or alkaline earth metal carbonate or hydrogencarbonate, triethylamine, pyridine or 1,8-diazabicyclundecene).

The reaction temperature usually ranges from room temperature to 200° C., preferably from about 25° C. to 150° C. The reaction is continued for 15 minutes to 48 hours. In usual, it may be completed within about 30 minutes to 15 hours.

The protective group of amino group may be an arbitrary one generally employed in the art. Examples thereof include optionally substituted alkoxycarbonyl groups (e.g., tert-butoxycarbonyl and 2,2, 2-trichloroethoxycarbonyl), optionally substituted aralkyloxycarbonyl groups (e.g., benzyloxycarbonyl, p-methoxybenzyloxycarbony and p-nitrobenzyloxycarbonyl), optionally substituted acyl groups (e.g., acetyl, methoxyacetyl, trifluoroacetyl, chloroacetyl, pivaloyl, formyl and benzoyl), optionally substituted alkyl groups and optionally substituted aralkyl groups (e.g., tert-butyl, benzyl, p-nitrobenzyl, p-methoxybenzyl and triphenylmethyl), ethers (e.g., metoxymethyl, tert-butoxymethyl, tetrahydropyranyl and 2,2,2-trichloroethoxymethyl) and silyl groups (e.g., trimethylsilyl, isopropyldimethylsilyl, tert-butyldimethylsilyl, tribenzylsilyl and tert-butyldiphenylsilyl).

When Y and Y¹ represent each an alkyl group having 1 to 6 carbon atoms, an alkoxymethyl group having 2 to 7 carbon atoms or a phenylalkyl group composed of an alkylene group having 1 to 6 carbon atoms with a phenyl group, the compound can be converted into the corresponding carboxylic acid by treating under acidic or basic conditions generally employed in the hydrolysis of carboxylates.

When Y¹ is a structure represented by the following formula:

—B(Y¹¹)Y¹²

the compound of the formula (IV) is reacted with the compound of the formula (VI) and then treated under acidic or basic conditions to thereby convert the reaction product into the corresponding carboxylic acid.

When deprotection is needed, the protective group is removed under appropriate procedure known in the art for the protective group used to thereby give the target compound of the formula (I).

The compound represented by the formula (VI) can be formed by removing Q′ from a compound represented by the following formula (VII):

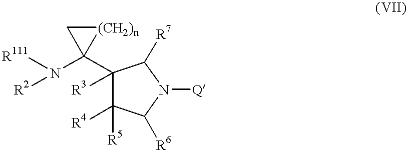

wherein R¹¹¹ represents a hydrogen atom, an alkyl group having 1 to 6 carbon atoms or a protective group of amino group;

R² represents a hydrogen atom or an alkyl group having l,to 6 carbon atoms, provided that the alkyl group may have one or more substituents selected from the group consisting of a hydroxyl group, a halogen atom and an alkoxy group having 1 to 6 carbon atoms;

R³ and R⁵, each represents a hydrogen atom;

R⁴ represents a hydroxyl group, a halogen atom, a carbamoyl group, an alkyl group having 1 to 6 carbon atoms, an alkoxyl group having 1 to 6 carbon atoms or an alkylthio group having 1 to 6 carbon atoms, provided that the alkyl group may have one or more substituents selected from the group consisting of a hydroxyl group, a halogen atom and an alkoxy group having 1 to 6 carbon atoms; and

R⁴ and the substituent on the pyrrolidine ring of the following formula:

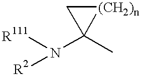

are located in the cis-configuration (as a matter of course, the substituents R³ and R⁵ are located at the cis-configuration too);

R⁶ and R⁷, each independently represents a hydrogen atom or an alkyl group having 1 to 6 carbon atoms;

n is an integer of from 1 to 3; and

Q represents a protective group for amino group selected from the group consisting of optionally substituted alkoxycarbonyl groups, optionally substituted aralkyloxycarbonyl groups, optionally substituted acyl groups, optionally substituted alkyl groups, optionally substituted aralkyl groups and substituted silyl groups.

The compound represented by the formula (VII) may present as salts or hydrates thereof or hydrates of the salts. Examples of the acid addition salts include inorganic acid salts and organic acid salts. More particularly speaking, inorganic acid salts (e.g., hydrochloride, sulfate, nitrate, hydrobromide, hydrolodide and phosphate) and organic acid salts (e.g., sulfonates such as methanesulfonate, benzenesulfonate and toluenesulfonate, and carboxylates such as acetate, citrate, maleate, fumarate and lactate) are exemplified.

When R¹¹¹ and Q′ are both protective group of amino group, they may be either the same or different from each other. To produce the compound (I), it is advantageous that these protective groups for amino group are those which are removed under different reaction conditions.

Examples of the protective groups for R¹¹¹ and Q′ include optionally substituted alkoxycarbonyl groups, optionally substituted aralkyloxycarbonyl groups, optionally substituted acyl groups, optionally substituted alkyl groups, optionally substituted aralkyl groups and substituted silyl groups.

Particular examples thereof include optionally substituted alkoxycarbonyl groups (e.g., tert-butoxycarbonyl and 2,2,2-trichloroethoxycarbonyl), optionally substituted aralkyloxycarbonyl groups (e.g., benzyloxycarbonyl-p-methoxybenzyloxycarbony and p-nitrobenzyloxycarbonyl), optionally substituted acyl groups (e.g., acetyl, methoxyacetyl, trifluoroacetyl, chloroacetyl, pivaloyl, formyl and benzoyl), optionally substituted alkyl groups and optionally substituted aralkyl groups (e.g., tert-butyl, benzyl, p-nitrobenzyl, p-methoxybenzyl and triphenylmethyl), ethers (e.g., metoxymethyl, tert-butoxymethyl, tetrahydropyranyl and 2,2,2-trichloroethoxymethyl) and substituted silyl groups (e.g., trimethylsilyl, isopropyldimethylsilyl, tert-butyldimethylsilyl, itribenzylsilyl and tert-butyldiphenylsilyl).

The compound of the formula (VII) can be produced as the cis-compound by forming a compound (pyrroline derivative) wherein the carbon atom to which the substituent R⁴ is bonded and the adjacent carbon atom is bonded via a double bond followed by catalytic reduction. Alternatively, the cis-compound can be produced by once forming a compound wherein the substituent R⁴ and the substituent moiety having the cyclic structure are located at the trans-configuration and then inverting the configuration of the substituent R⁴.

Because of having potent antimicrobial effects, the compounds of the present invention are usable as drugs for human being, animals and fishes or preservatives for agricultural chemicals and foods.

When used as drugs for human being, the compounds of the present invention are administered to an adult in a dose of from 50 mg to 1 g per day, preferably from 100 mg to 300 mg per day.

When used as drugs for animals, the dose of the compounds of the present invention varies depending on the purpose (therapeutic use or prevention, etc.) of the administration, the type and size of the animal to be treated, the pathogenic bacterium and the severity of the infection. In general, a daily dose ranges from 1 mg to 200 mg per kg body weight, preferably from 5 mg to 100 mg per kg.

Such a compound is administered in the above daily dose once to 4 times in a day. If necessary, it may be administered in a dose exceeding the above-mentioned level.

The compounds of the present invention are efficacious against infective microorganisms over a wide range and can treat, prevent or ameliorate diseases caused by these microorganisms.

Examples of the bacteria and bacterium-like microorganisms against which the compounds of the present invention are efficacious include Staphylococcus, Streptococcus pyogenes, hemolytic streptococcus, enterococcus, pneumococcus, the genus peptostreptococcus, Neisseria gonorrhoeae, Escherichia coli, the genus citrobacter, the genus shigella, Klebsiella pneumoniae, the genus enterobacter, the genus serratia, the genus proteus, Pseudomonas aeruginosa, influenza virus, the genus acinetobacter, the genus campylobacter and Chlamydia trachomatis.

Examples of the diseases induced by these pathogenic microorganisms include folliculitis, furuncle, carbuncle, erysipelas, phlegmon, lymphantitis, lymphnoditis, panaritium (felon), subcutaneous tumor, hidrosadenitis, aggregated acne, infectious atheroma, anal abscess, mastitis, superfacial secondary infection such as trauma, burn and operative wound, pharyngolaryngitis, acute bronchitis, tonsillitis, chronic bronchitis, bronchiectasis, diffuse bronchiolitis, secondary infection in chronic respiratory diseases, pneumonia, pyelonephritis, cystitis, prostatitis, epididymitis, gonococcal urethritis, non-gonococcal urethritis, cholecystitis, cholangitis, bacillary dysentery, enteritis, uterine adnexitis, intrauterine infection, bartholinitis, tarsitis, hordeolum, dacryocystitis, tarsadenitis, corneal ulcer, oititis media, sinusitis, periodontium inflammation, pericoronitis, jaw inflammation, peritonitis, endocarditis, sepsis, meningitis and skin infectious diseases.

The compounds of the present invention are also efficacious against microorganisms causing infectious diseases in animals, for example, escherichia, salmonella, pasteurella, hemophilus, bordetella, staphylococcus and mycoplasma. Particular examples of the diseases caused by these microorganisms include bird diseases (e.g., E. coli infection, pullorum disease, avian paratyphoid, avian cholera, infectious coryza, staphylococcus infection and mycoplasma infection), swine diseases (e.g., E. coli infection, salmonellosis, pasteurellosis, hemophilus infection, atrophic rhinitis, exudative superfacial inflammation and mycoplasma infection), bovine diseases (e.g., E. coli infection, salmonellosis, hemorrhagic septicemia, mycoplasma infection, bovine pleuropneumonia and bovine mastitis), canine diseases (e.g., coliemia, salmonellosis, hemorrhagic septicemia, uterine empyema and cystitis) and feline diseases (e.g., exudative pleurisy, cystitis, chronic rhinitis, Haemophilus infection, kitten diarrhea and mycoplasma infection).

Antimicrobial agents comprising the compounds of the present invention may be processed into appropriate preparations depending on the administration method by using various processes commonly employed in the art. Examples of the dosage forms of the antimicrobial preparations for oral use containing the compounds of the present invention as the principal agent include tablets, dusts, granules, capsules, solutions, syrups, elixirs and oily and aqueous suspension.

When employed as injections, the preparations may contain stabilizers, antiseptics and solubilizers. A solution optionally containing these auxiliary components may be packed into a container and processed into a solid preparation by freeze-drying, etc. to give a product to be prepared before suing. Such a preparation may be packed in a container in a single dose. Alternatively, it may be packed in a single container in a number of doses.

Examples of external preparations include solutions, suspensions, emulsions, ointments, gels, creams, lotions and sprays.

Solid preparations may be produced by blending the active compounds with pharmaceutically acceptable additives appropriately selected from among excipients, fillers, binders, disintegration agents, dissolution accelerators, moistening agents, lubricants, etc. followed by processing.

Examples of liquid preparations include solutions, suspensions and emulsions. These preparations may contain suspending agents, emulsifiers, etc. as additives.

The methods for administering the compounds of the present invention to animals include an oral administration method comprising directly adding to feed; an oral administration method comprising once preparing solutions followed by direct administration or addition to drinking water or feed; and an injection method.

To administer to animals, the compounds of the present invention can be processed into dusts, fine subtilaes, soluble dusts, syrups, solutions or injections by the techniques commonly employed in the art.

Next, formulation examples will be given.

Formulation Example 1 [Capsule]:

| | | |
|---|---|---|
| Compound of Example 3 | 100.0 | mg |
| Corn starch | 23.0 | mg |
| CMC calcium | 22.5 | mg |
| Hydroxymethylcellulose | 3.0 | mg |
| Magnesium stearate | 1.5 | mg |
| Total | 150.0 | mg |

Formulation Example 2 [Solution]:

| | | |
|---|---|---|
| Compound of Example 5 | 1-10 | g |
| Acetic acid or sodium hydroxide | 0.5-2 | g |
| Ethyl parahydoxybenzoate | 0.1 | g |
| Purified water | 87.9-98.4 | g |
| Total | 100 | g |

Formulation Example 3 [Dust to be added to animal feed]:

| | | |
|---|---|---|
| Compound of Example 7 | 1-10 | g |
| Corn starch | 98.5-89.5 | g |
| Soft silicic anhydride | 0.5 | g |
| Total | 100 | g |

Formulation Example 2 [Solution]:

| | | |
|---|---|---|
| Compound of Example 5 | 1-10 | g |
| Acetic acid or sodium hydroxide | 0.5-2 | g |
| Ethyl parahydoxybenzoate | 0.1 | g |
| Purified water | 87.9-98.4 | g |
| Total | 100 | g |

Formulation Example 3 [Dust to be added to animal feed]:

| | | |
|---|---|---|
| Compound of Example 7 | 1-10 | g |
| Corn starch | 98.5-89.5 | g |
| Soft silicic anhydride | 0.5 | g |
| Total | 100 | g |

### BEST MODE FOR CARRYING OUT INVENTION

To further illustrate the present invention in greater detail, and not by way of limitation, the following Examples will be given. The antimicrobial activity of each target compound was evaluated in accordance with the standard method specified by Japan Society of Chemotherapy and expressed in MIC (μg/ml).

### Referential Example 1-1

### Ethyl 3-(1-tert-butoxycarbonylaminocyclopropyl)propiolate

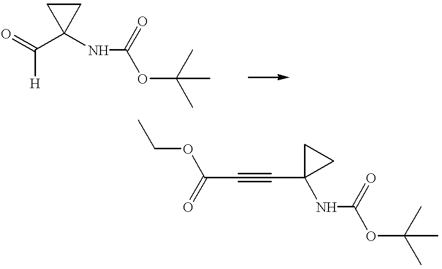

Under a nitrogen atmosphere, chloromethyltrimethyl-phosphonium chloride (5.156 g, 14.85 mmol) was suspended in dry tetrahydrofuran (30 ml). After cooling the suspension to give an internal temperature of −55° C., a 1.68 M solution of n-butyllithium in n-hexane (8.87 ml, 14.90 mmol) was dropped thereinto over 5 minutes. Then the reaction suspension was stirred under ice cooling for 30 minutes and then at room temperature for additional 3 hours followed by cooling to give an internal temperature of −55° C. Into this reaction suspension was dropped a solution of 1-tert-butoxycarbonylaminocyclopropane carbaldehyde (2.49 g, 13.50 mmol) in dry tetrahydrofuran (10 ml) over 10 minutes and the resultant mixture was stirred at −50° C. for 1 hour and then under ice cooling for additional 30 minutes. The reaction suspension was cooled to −78° C. and a 1.68 M solution of n-butyllithium in n-hexane (17.68 ml, 29.70 mmol) was dropped thereinto over 10 minutes followed by stirring at −78° C. for 20 minutes. Next, ethyl chloroformate (1.61 ml, 16.88 mmol) was dropped into this reaction suspension followed by stirring at −78° C. for 1.5 hours and then under ice cooling for 1 hour. Under ice cooling, a saturated aqueous solution of sodium chloride (30 ml) was added to the reaction suspension and the organic Iayer was separated. The aqueous layer was extracted with diethyl ether (30 ml×2) and the combined organic layer was washed with a saturated aqueous solution of sodium chloride (30 ml) and dried over anhydrous magnesium sulfate. After filtering, the filtrate was concentrated under reduced pressure and the residue was subjected to flash silica gel column chromatography (eluent: n-hexane:ethyl acetate=5:1) to give 2.178 g (63.9%) of the title compound as a colorless oily substance.

¹H—NMR (400 MHz, CDCl₃) δ: 5.04 (brs, 1H), 4.27 (q, J =7.16 Hz, 2H), 1.44 (s, 9H), 1.28 (t, J=7.16 Hz, 3H), 1.15 (m, 2H),1.06 (m, 2H).

### Referential Example 1-2

### Ethyl 1-benzyl-4-(1-tert-butoxycarbonylaminocyclopropyl)-3-pyrroline-3-carboxylate

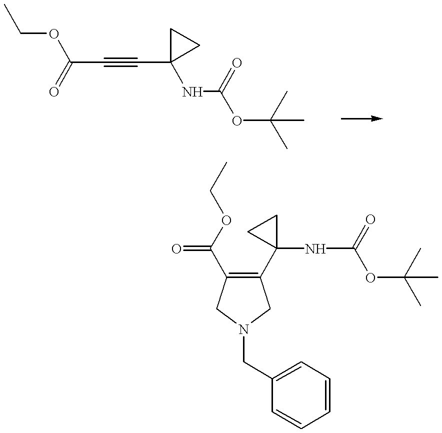

N-Benzyl-N-(n-butoxymethyl)trimethylsilylmethylamine (2.006 g, 7.176 mmol) and ethyl 3-(1-tert-butoxycarbonylaminocyclopropyl)propiolate (1.136 g, 4.485 mmol) were dissolved in dry dichloromethane (9 ml). While stirring at room temperature, a 1.0 M solution of trifluoroacetic acid in dichloromethane (0.72 ml, 0.72 mmol) was added thereto and the liquid reaction mixture was stirred for 3 hours. Then a saturated aqueous solution of sodium bicarbonate (20 ml) was added to the liquid reaction mixture followed by extraction with dichloromethane (20 ml×3). The combined organic layer was washed with a saturated aqueous solution of sodium chloride (30 ml) and dried over anhydrous magnesium sulfate. After filtering, the filtrate was concentrated under reduced pressure and the residue was subjected to flash silica gel column chromatography (eluent: chloroform) to give 1.449 g (83.6%) of the title compound as a colorless oily substance.

¹H—NMR (400 MHz, CDCl₃) δ: 7.40-7.11 (m, 5H), 5.17 (brs,. 1H), 4.12 (q, J=6.83 Hz, 2H), 3.85 (m, 2H), 3.72 (m, 2H), 3.67 (s, 2H), 1.44 (s, 9H), 1.24 (t, J=6.83 Hz, 3H), 1.14 (m, 2H), 1.01 (m, 2H).

### Referential Example 1-3

### Ethyl cis-1-benzyl-4-(1-tert-butoxycarbonylaminocyclopropyl)-pyrrolidine-3-carboxylate

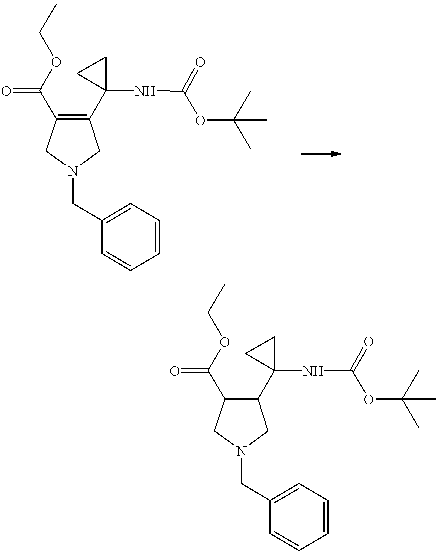

Under a nitrogen gas stream, bis(bicyclo[2.2.1]hepta-2,5-diene)rhodium (I) perchlorate (54.5 mg, 0.14 mmol) and 1,2-bis(diphenylphosphino)ethane (67.4 mg, 0.17 mmol) were dissolved in degassed methanol (25 ml) and stirred at room temperature for 10 minutes. To this catalyst solution was added a solution of ethyl 1-benzyl-4-(1-tert-butoxycarbonylaminocyclopropyl)-3-pyrroline-3-carboxylate (1.090 g, 2.820 mmol) in dry and degassed methanol (15 ml). The obtained liquid reaction mixture was then stirred under a hydrogen atmosphere (1 kg/cm²) at room temperature for 2.5 hours. After adding active carbon (1 g), the liquid reaction mixture was allowed to stand at room temperature for 30 minutes and then filtered through celite (washed with methanol). The filtrate was concentrated under reduced pressure and the residue was subjected to flash silica gel column chromatography (eluent: n-hexane:ethyl acetate=5:1) to give 1.071 g (97.8%) of the title compound as colorless crystals.

¹H—NMR (400 MHz, CDCl₃) δ: 7.40-7.19 (m, 5H), 5.07 (brs, 1H), 4.13 (q, J=7.33 Hz, 2H), 3.63 (s, 2H), 2.87 (m, 1H), 2.67 (m, 1H), 2.54 (m, 1H), 2.35 (m, 1H), 2.15 (m, 1H), 1.79 (m, 1H), 1.46 (s, 9H), 1.23 (t, J=7.33 Hz, 3H), 0.85 (m, 2H), 0.69 (m, 2H).

### Referential Example 1-4

### cis-1-Benzyl-4-(1-tert-butoxycarbonylaminocyclopropyl)-3-hydroxymethylpyrrolidine

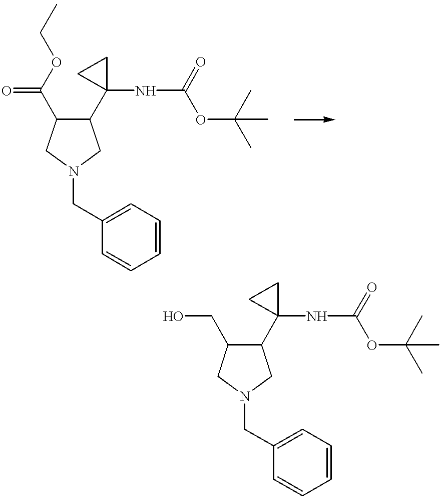

Under a nitrogen gas stream, lithium aluminum hydride (195.6 mg, 5.135 mmol) was suspended in dry tetrahydrofuran (40 ml). Under stirring at −15° C., a solution of ethyl cis-1-benzyl-4-(1-tert-butoxycarbonylaminocyclopropyl)-pyrrolidine-3 -carboxylate (1.001 g, 2.577 mmol) in dry tetrahydrofuran (10 ml) was dropped thereinto over 15 minutes. After stirring the reaction suspension under ice cooling for 3.5 hours, cooling water (5 ml) was slowly added thereto and the mixture was stirred at room temperature for additional 15 minutes. The reaction suspension was filtered through celite (washed with diethyl ether). The filtrate was concentrated under reduced pressure and dried to give 833.9 mg (93.4%) of the title compound as a colorless oily substance.

¹H—NMR (400 MHz, CDCl₃) δ: 7.39-7.00 (m, 5H), 5.10 (brs, 1H), 3.69 (m, 2H), 3.58 (s, 2H), 2.99 (m, 1H), 2.61 (m, 1H), 2.51 (m, 1H), 2.27 (m, 1H), 2.00 (m, 1H), 1.94 (brs, 1H), 1.74 (m, 1H), 1.42 (s, 9H), 0.90 (m, 1H), 0.74-0.61 (m, 3H).

### Referential Example 1-5

### cis-4-(1-tert-Butoxycarbony-lminocyclopropyl)-3-hydroxymethylpyrrolidine

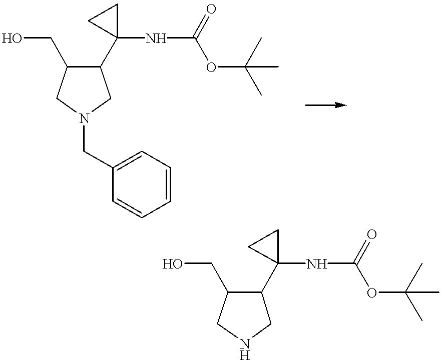

cis-1-Benzyl-4-(1-tert-butoxycarbonylamino-cyclopropyl)-3 -hydroxymethylpyrrolidine (820.1 mg, 2.376 mmol) was dissolved in methanol (50 ml). After adding 5% palladium-carbon catalyst (moisture content: 55.6%, 750 mg), the mixture was stirred under elevated hydrogen pressure (4.5 kg/cm²) over day and night. After filtering off the catalyst through celite (washed with methanol), the filtrate was concentrated under reduced pressure to give 578.8 mg (91.0%) of the title compound as a white amorphous substance.

¹H—NMR (400 MHz, CDCl₃) δ: 5.05 (brs, 1H), 3.72 (m, 2H), 3.15 (m, 2H), 2.82 (m, 2H), 2.29 (m, 1H), 1.94 (br, 2H), 1.76 (m, 1H), 1.42 (s, 9H), 0.92 (m, 2H), 0.82 (m, 1H), 0.61 (m, 1H).

### EXAMPLE 1

### 5-Amino-7-[cis-4-(1-aminocycloproiyl)-3-hydroxymethyl-1-pyrrolidinyl]-1-cyclopropyl-6-fluoro-1,4-dihydro-8-methyl-4-oxoquinoline-3-carboxylic acid

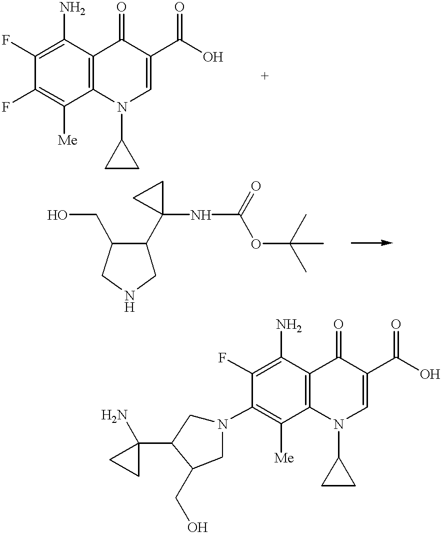

cis-4-(1-tert-Butoxycarbonylaminocyclopropyl)-3-hydroxymethylpyrrolidine (550.1 mg, 2.146 mmol) was dissolved in dimethyl sulfoxide (15 ml) and triethylamine (3.5 ml) and 5-amino-1-cyclopropyl-6,8-difluoro-1,4-dihydro-8-methyl-4-oxoquinoline-3-carboxylic acid (300.2 mg, 1.020 mmol) were added thereto. Then the mixture was stirred under a nitrogen atmosphere in an oil bath at 150° C. for 22 hours. After allowing to cool, dimethyl sulfoxide was evaporated. The residue was dissolved in chloroform (100 ml), washed successively with a 10% aqueous solution of citric acid (100 ml) and a saturated aqueous solution of sodium chloride (50 ml). The organic layer was dried over anhydrous magnesium sulfate. After filtering, the filtrate was concentrated under reduced pressure. Under ice cooling, conc. hydrochloric acid (10 ml) was dropped into the residue followed by stirring for 1 hour. The liquid reaction mixture was washed with dichloromethane (20 ml×4) and the pH value of the aqueous layer was adjusted to 12 with a 15% aqueous solution of sodium hydroxide followed by washing with dichloromethane (20 ml×2). The pH value of this aqueous solution was adjusted to 7.2 with 1 N hydrochloric acid followed by extraction with chloroform (100 ml×4). The combined organic layer was dried over anhydrous magnesium sulfate and filtered. The filtrate was concentrated under reduced pressure and the crude product thus obtained was recrystallized from a 2-propanol/diisopropyl ether system. The crystals thus obtained were dried under reduced pressure at 70° C. for 18 hours to give 112.4 mg (25.6%) of the title compound as yellow crystals.

Melting point: 158.8-159.9° C. (decomp.).

¹H—NMR (400 MHz, 0.1 N—NaOD) δ: 8.39 (s, 1H), 3.99 (m, 1H), 3.80 (dd, J=11.23, 5.37 Hz, 1H), 3.62 (m, 2H), 3.51 (d, J=7.32, 2H), 3.41 (t, J=7.81 Hz, 1H), 2.45 (m, 1H), 2.37 (s, 3H), 1.71 (q, J=7.81, 1H), 1.18 (m, 2H), 0.74 (m, 1H), 0.70 (m, 1H), 0.55 (m, 4H).

Elemental analysis data: as C₂₂H₂₇FN₄O₄ calcd.: C, 61.31; H, 6.32; N. 13.02 found: C, 61.25; H, 6.32; N, 12.74.

### Referential Example 2-1

### 4-(S)-(1-Ethoxycarbonylcyclopropyl)-3-(R)-fluoro-1-[1-(S)-phenylethyl]-2-pyrrolidone

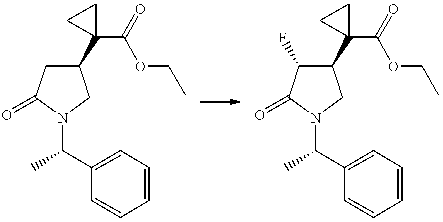

Under a nitrogen atmosphere, diisopropylamine (3.99 ml, 30.4 mol) was dissolved in dry tetrahydrofuran (50 ml). After cooling the solution to −78° C., a 1.68 M solution of n-butyllithium in n-hexane (18.1 ml, 30.4 mmol) was dropped thereinto over 10 minutes. Then the liquid reaction mixture was stirred at −10° C. for 20 minutes and cooled to −78° C. Next, a solution of 4-(S)-(1-ethoxycarbonylcyclopropyl)-1-[1-(S)-phenylethyl]-2-pyrrolidone (7.052 g, 23.40 mmol) in dry tetrahydrofuran (30 ml) was dropped thereinto over 15 minutes. The liquid reaction mixture was stirred at −78° C. for 1 hour. Then a solution of N-fluorobenzenedisulfonimide (11.81 g, 37.44 mmol) in dry tetrahydrofuran (60 ml) was dropped thereinto at the same temperature over 25 minutes. The liquid reaction mixture was stirred at −78° C. for 2 hours and then heated to room temperature followed by stirring for additional 20 minutes. Under ice cooling, a saturated aqueous solution of ammonium chloride (200 ml) was added to the liquid reaction mixture. The organic layer was separated and the aqueous layer was extracted with diethyl ether (200 ml×2). The combined organic layer was washed with water (200 ml×3) and dried over anhydrous magnesium sulfate. After filtering, the filtrate was concentrated under reduced pressure and the residue was subjected to flash silica gel column chromatography (eluent: n-hexane:ethyl acetate=3:1) to give 5.276 g (70.6%) of the title compound as a colorless oily substance.

¹H—NMR (400 MHz, CDCl₃) δ: 0.76-0.81 (1H, m), 0.89-0.93 (1H, m), 1.09 (3H, t, J 6.84 Hz), 1.24-1.34 (2H, m), 1.58 (3H, d, J=7.33 Hz), 2.23 (1H, dq, J 28.32, 8.30 Hz), 2.88-2.93 (1H, m), 3.48 (1H, t, J=9.28 Hz), 3.92-4.08 (2H, m), 5.14 (1H, dd, J 53.71, 7.81 Hz), 5.54 (1H, q, J=7.33 Hz), 7.27-7.34 (5H, m).

### Referential Example 2-2

### 4-(S)-(1-Ethoxycarbonylcyclovropyl)-3-(S)-fluoro-1-[1-(S)-phenylethyl]-2-pyrrolidone

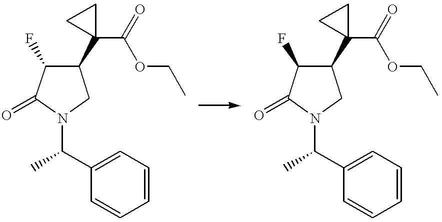

Under a nitrogen atmosphere, diisopropylamine (7.22 ml, 51.52 mmol) was dissolved in dry tetrahydrofuran (100 ml). After cooling the solution to −78° C., a 1.68 M solution of n-butyllithium in n-hexane (28.1 ml, 47.21 mmol) was dropped thereinto over 15 minutes. Then the liquid reaction mixture was stirred at 0° C. for 10 minutes and cooled to −78° C. Next, a solution of 4-(S)-(1-ethoxycarbonylcyclopropyl)-3-(R)-fluoro-1-[1-(S)-phenylethyl]-2-pyrrolidone (13.72 g, 42.96 mmol) in dry tetrahydrofuran (40 ml) was dropped thereinto over 20 minutes. The liquid reaction mixture was stirred at −78° C. for 20 minutes. Then a solution of 2,6-di-tert-butylphenol (10.63 g, 51.52 mmol) in dry tetrahydrofuran (40 ml) was dropped thereinto over 20 minutes. The liquid reaction mixture was stirred at −78° C. for 10 minutes and then heated to room temperature. Under ice cooling, a saturated aqueous solution of ammonium chloride (200 ml) was added to the liquid reaction mixture. The organic layer was separated and the aqueous layer was extracted with diethyl ether (200 ml×2). The combined organic layer was washed with water (400 ml ×2) and dried over anhydrous magnesium sulfate. After filtering, the filtrate was concentrated under reduced pressure and the residue was subjected to flash silica gel column chromatography (eluent: n-hexane:ethyl acetate=3:1) to give 10.19 g (74.2%) of the title compound as a colorless oily substance.

¹H—NMR (400 MHz, CDCl₃) δ: 0.57-0.63 (1H, m), 0.78-0.84 (1H, m), 1.07-1.13 (1H, m), 1.26 (3H, t, J=7.09 Hz), 1.23-1.29 (1H, m), 1.54 (3H, d, J=7.32 Hz), 2.59 (1H, t, J=9.77 Hz), 3.05 (1H, dq, J=28.81, 8.30 Hz), 3.25 (1H, t, J=9.77 Hz), 4.00-4.16 (2H, m), 5.15 (1H, dd, J=52.73, 6.35 Hz), 5.53 (1H, q, J=7.32 Hz), 7.27-7.38 (5H, m).

### Referential Example 2-3

### 4-(S)-(1-Ethoxycarbonylcyclopropyl)-3-(S)-fluoro-1-[1-(S)-phenylethyl]-2-pyrrolidinthione

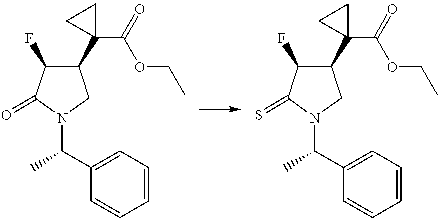

4-(S)-(1-Ethoxycarbonylcyclopropyl)-3-(S)-fluoro-1-[1-(S)-phenylethyl]-2-pyrrolidone (6.86 g, 21.48 mmol) was dissolved in dry toluene (100 ml). After adding Lawesson reagent (5.21 g, 12.89 mmol), the mixture was heated at 60° C. for 30 minutes. After allowing the liquid reaction mixture to cool, toluene was evaporated under reduced pressure and the residue was subjected to flash silica gel column chromatography (eluent: n-hexane:ethyl acetate=4:1) to give 6.49 g (90.1%) of the title compound as a pale yellow oily substance.

¹H—NMR (400 MHz, CDCl₃) δ: 0.59-0.66 (1H, m), 0.86-0.92 (1H, m), 1.08-1.15 (1H, m), 1.20 (3H, t, J=7.33 Hz), 1.24-1.31 (1H, m), 1.60 (3H, d, J=7.32 Hz), 2.85 (1H, dd, J=11.23, 9.28 Hz), 3.16 (1H, dq, J=30.27, 8.30 Hz), 3.50 (1H, dd, J=11.23, 9.28 Hz), 4.04-4.15-(2H, m), 5.32 (1H, dd, J=52.73, 5.38 Hz), 6.28-6.34 (1H, m), 7.30-7.41 (5H, m).

### Referential Example 2-4

### 4-(S)-(1-Ethoxycarbonylcyclopropyl)-3-(S)-fluoro-1-[1-(S)-phenylethyl]pyrrolidine

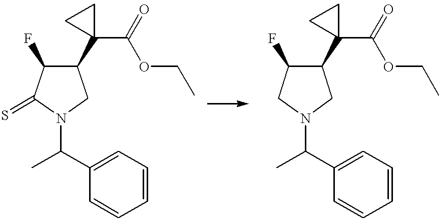

4-(S)-(1-Ethoxycarbonylcyclopropyl)-3-(S)-fluoro-1-[1-(S)-phenylethyl]-2-pyrrolidinthione (6.49 g, 19.35 mrnol) was dissolved in dry tetrahydrofuran (150 ml). After adding Raney nickel catalyst (15 mnl), the mixture was stirred at room temperature for 30 minutes. After eliminating the catalyst by filtering through celite (washed with tetrahydrofuran), the filtrate was concentrated under reduced pressure. The residue was dissolved in diethyl ether (200 ml) and the obtained solution was washed with a 10% aqueous solution of ammonia (200 ml×2) and a saturated aqueous solution of sodium chloride (150 ml) and dried over anhydrous sodium sulfate. After filtering, the filtrate was concentrated under reduced pressure to give 5.08 g (86.0%) of the title compound as a colorless oily substance.

¹H—NMR (400 MHz, CDCl₃) δ: 0.54-0.60 (1H, m), 0.95-1.08 (2H, m), 1.22 (3H, t, J=7.33 Hz), 1.25-1.32 (1H, n), 1.35 (3H, d, J 6.35 Hz), 1.99 (1H, t, J=9.28 Hz), 2.42 (1H, t, J=8.30 Hz), 2.63 (1H, ddd, J=33.21, 11.72, 1.95 Hz), 2.99 (1H, dm, J=28.32 Hz), 3.25-3.37 (2H, m), 4.03-4.16 (2H, m), 5.33 (1H, dm, J=55.67 Hz), 7.21-7.36 (5H, m).

### Referential Example 2-5

### 1-Benzyloxycarbonyl-4-(S)-(1-ethoxycarbonylcyclopropyl)-3-(S)-fluorotyrrolidine

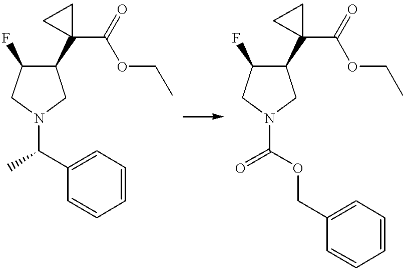

4-(S)-(1-Ethoxycarbonylcyclopropyl)-3-(S)-fluoro-1-[1-(S)-phenylethyl]pyrrolidine (5.08 g, 16.63 mmol) was dissolved in dry dichloromethane (50 ml). Under ice-cooling, benzyl chloroformate (3.56 ml, 25.0 mmol) was dropped into this solution. Then the liquid reaction mixture was heated under reflux for 1 hour and dichloromethane was evaporated under reduced pressure. The residue was subjected to flash silica gel column chromatography (eluent: n-hexane:ethyl acetate=3:1) to give 4.67 g (83.7%) of the title compound as a colorless oily substance.

¹H—NMR (400 MHz, CDCl₃) δ: 0.71-0.78 (1M, m), 1.11-1.23 (2H, m), 1.24 (3H, t, J=6.84 Hz), 1.29-1.37 (1H, m), 2.93-3.00 (1H, m), 3.10 (1H, dm, J=34.67 Hz), 3.54-3.84 (2H, m), 4.09-4.18 (2H, m), 5.14 (2H, s), 5.34 (1H, ddm, J=53.71, 16.6 Hz), 7.29-7.38 (5H, m).

### Referential Example 2-6

### 1-[1-Benzyloxycarbonyl-4-(S)-fluoro-3-(S)-pyrrolidinyl]-cyclopropanecarboxylic acid

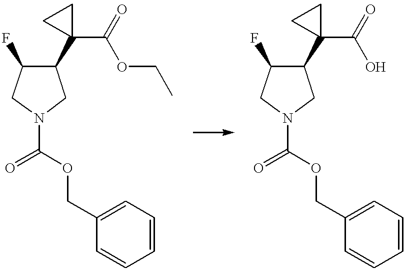

1-Benzyloxycarbonyl-4-(S)-(1-ethoxycarbonyl-cyclopropyl)-3-(S)-fluoropyrrolidine (4.67 g, 13.92 mmol) was dissolved in ethanol (50 ml). Then a 1 N aqueous solution of sodium hydroxide (50 ml) was dropped into this solution. Then the liquid reaction mixture was stirred at 40° C. for 1.5 hours and ethanol was evaporated under reduced pressure. Water (50 ml) was added to the residue followed by washing with chloroform (100 ml). The aqueous layer was separated and acidified by dropping 1 N hydrochloric acid thereinto. Next, it was extracted successively with chloroform (200 ml×2) and diethyl ether (100 ml). The combined organic layer was dried over anhydrous sodium sulfate. After filtering, the filtrate was concentrated under reduced pressure to give 3.94 g (92.1%) of the title compound as a colorless amorphous substance.

¹H—NMR (400 MHz, CDCl₃) δ: 0.79-0.89 (1H, m), 1.18-1.35 (2H, m), 1.37-1.47 (1H, m), 2.90-3.18 (2H, m), 3.50-3.84 (3H, m), 5.13 (2H, s), 5.31 (1H, ddm, J=53.22, 15.13 Hz), 7.26-7.42 (5H, m).

### Referential Example 2-7

### 1-Benzyloxycarbonyl-4-(R)-(1-tert-butoxycarbonylaminocyclopropyl)-3-(S)-fluoropyridine

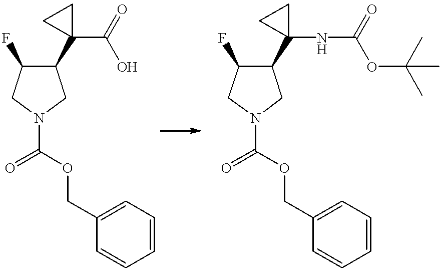

1-[1-Benzyloxycarbonyl-4-(S)-fluoro-3-(S)-pyrrolidinyl]cyclopropanecarboxylic acid (3.22 g, 10.48 mmol) was dissolved in dry acetonitrile (80 ml). After adding N,N′-carbonyldiimidazole (2.55 g, 15.73 mmol), the liquid reaction mixture was stirred at room temperature for 30 minutes. Next, ammonia gas was bubbled thereinto at the same temperature. Then the liquid reaction mixture was concentrated under reduced pressure. Water (80 ml) was added to the residue followed by extraction with chloroform (80 ml ×2). The combined organic layer was dried over anhydrous sodium sulfate. After filtering, the filtrate was concentrated under reduced pressure. The residue was dissolved in tert-butyl alcohol (100 ml) and lead tetraacetate (7.93 g, 15. 70 mmol) was added thereto. After heating under reflux for 30 minutes, the liquid reaction mixture was allowed to cool and diethyl ether (50 ml) and sodium hydrogencarbonate (10 g) were added thereto. Then the mixture was stirred at room temperature for 10 minutes and filtered. The filtrate was concentrated under reduced pressure. After adding ethyl acetate (150 ml) to the residue, the mixture was washed with a saturated aqueous solution of sodium bicarbonate and dried over anhydrous sodium sulfate. After filtering, the filtrate was concentrated under reduced pressure and the residue was subjected to flash silica gel column chromatography (eluent: n-hexane:ethyl acetate=3:2) to give 3.216 g (81.2%) of the title compound as a colorless oily substance.

¹H—NMR (400 MHz, CDCl₃) δ: 0.65-0.74 (1H, m), 0.70-0.84 (1h, m), 0.85-1.00 (2H, m), 1.42 (9H, s), 2.21 (1H, ddm, J=80.57, 36.14 Hz), 3.08-3.24 (2H, m), 3.48-3.84 (3H, m), 5.02 (1H, brs), 5.13 (2H, s), 5.15 (1H, brd, J=53.72 Hz), 7.28-7.38 (5H, m).

### EXAMPLE 2

### 5-Amino-7-[4-(R)-(1-aminocyclopropyl)-3-(S)-fluoro-1-pyrrolidinyl]-6-fluoro-1-[2-(S)-fluoro-1-(R)-cyclopropyl1-1,4-dihydro-8-methyl-4-oxoquinoline-3-carboxylic acid hydrochloride

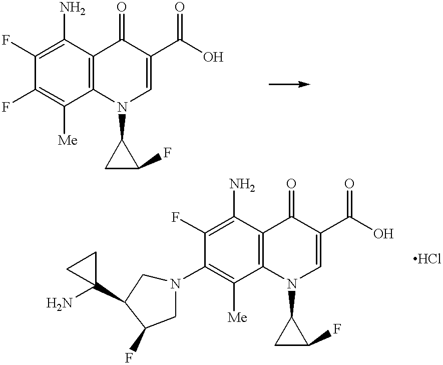

1-Benzyloxycarbonyl-4-(R)-(1-tert-butoxycarbonylaminocyclopropyl)-3-(S)-fluoropyrrolidine (1.43 g, 3.78 mmol) was dissolved in ethanol (60 ml). After adding 5% palladium-carbon catalyst (moisture content: 55.6%, 1.5 g), the mixture was stirred under a hydrogen atmosphere for 3 hours. After filtering off the catalyst through celite (washed with methanol), the filtrate was concentrated under reduced pressure. The residue thus obtained was dissolved in dimethyl sulfoxide (12 ml) and 5-amino-6,7-difluoro-1-[2-(S)-fluoro-1-(R)-cyclopropyl]-1,4-dihydro-8-methyl-4-oxoquinoline-3-carboxylic acid (1.18 g, 3.78 mmol) and triethylamine (3 ml) were added thereto. Then the mixture was stirred under a nitrogen atmosphere at 130° C. for 3 days. After allowing to cool, dimethyl sulfoxide was evaporated. The residue was dissolved in chloroform (80 ml), washed successively with a 10% aqueous solution of citric acid (80 ml) and a saturated aqueous solution of sodium chloride (100 ml). The organic layer was dried over anhydrous sodium sulfate. After filtering, the filtrate was concentrated under reduced pressure. The residue was subjected to flash silica gel column chromatography (eluent: chloroform:methanol=9:1) followed by concentration of the eluate under reduced pressure. Under ice cooling, conc. hydrochloric acid (10 ml) was dropped into the residue followed by stirring at room temperature for 50 minutes. After adding 1 N hydrochloric acid (30 ml), the liquid reaction mixture was washed with chloroform (50 ml×2) and its pH value was adjusted to 12.0 with an aqueous solution of sodium hydroxide followed by washing with chloroform (100 ml). The pH value of this aqueous solution was adjusted to 7.4 with 1 N hydrochloric acid followed by extraction with chloroform (150 ml×3). The combined organic layer was dried over anhydrous sodium sulfate and filtered. The filtrate was concentrated under reduced pressure and 1 N hydrochloric acid (2.0 ml) was dropped into the residue under ice cooling. After stirring at the same temperature for 5 minutes, the liquid reaction mixture was concentrated under reduced pressure (azeotropic distillation with ethanol, thrice). The residue was recrystallized from ethanol and dried under reduced pressure to give 230 mg (12.1%) of the title compound as a yellow powder.

Melting point: 213-218° C. (decomp.).

¹H—NMR (400 MHz, 0.1 N—NabD) δ: 0.55-0.71 (4H, m), 1-2.35 (1H, m), 3.32 (1H, t, J=8.79 Hz), 3.49 (1H, dd, J 25.88, 12.21 Hz), 3.85-3.97 (2H, m), 4.11 (1H, ddm, J=40.77, 12.45 Hz), 4.97 (1H, dm, J=70.31 Hz), 5.49 (1H, brd, J=55.18 Hz), 8.27 (1H, d, J=3.42 Hz).

Elemental analysis data: as C₂₁H₂₃F₃N₄O₃•HCl•1.25H₂O calcd.: C, 50.40; H, 5.33; N, 10.87 found: C, 50.45; H, 5.44; N. 11.21.

### EXAMPLE 3

### 5-Amino-7-[4-(R)-1-aminocyclopropyl)-3-(S)-fluoro-1-pyrrolidinyl]-6-fluoro-1-[2-(S)-fluoro-1-(R)-cyclopropyl]-1,4-dihydro-8-methoxy-4-oxoguinoline-3-carboxylic acid

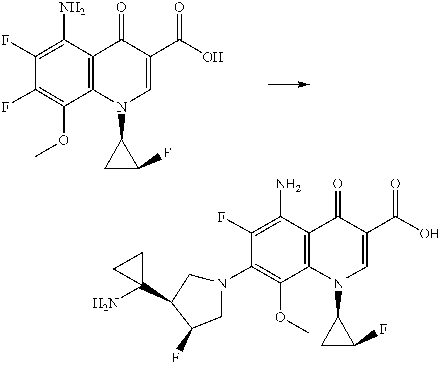

1-Benzyloxycarbonyl-4-(R)-(1-tert-butoxycarbonylaminocyclopropyl)-3-(S)-fluoropyrrolidine (400 mng, 1.06 inmol) was dissolved in ethanol (20 ml). After adding 5% palladium-carbon catalyst (moisture content: 55.6%, 500 mg), the mixture was stirred under a hydrogen atmosphere for 18 hours. After filtering off the catalyst through celite (washed with methanol), the filtrate was concentrated under reduced pressure. The residue thus obtained was dissolved in dimethyl sulfoxide (8 ml) and 5-amino-6,7-difluoro-1-(2-(S)-fluoro-1-(R)-cyclopropyl]-1,4-dihydro-8-methoxy-4-oxoquinoline-3-carboxylic acid (289 mg, 0.88 mmol) and triethylamine (2 ml) were added thereto. Then the mixture was stirred under a nitrogen atmosphere at 100° C. for 26 hours. After allowing to cool, dimethyl sulfoxide was evaporated. The residue was dissolved in chloroform (80 ml), washed with a 10% aqueous solution of citric acid (80 ml). The organic layer was dried over anhydrous sodium sulfate. After filtering, the filtrate was concentrated under reduced pressure. The residue was subjected to flash silica gel column chromatography (eluent: chloroform:methanol=9:1) followed by concentration of the eluate under reduced pressure. Under ice cooling, conc. hydrochloric acid (5 ml) was dropped into the residue followed by stirring at room temperature for 20 minutes. After adding 1 N hydrochloric acid (30 ml), the liquid reaction mixture was washed with chloroform (50 ml×2) and its pH value was adjusted to 12.0 with an aqueous solution of sodium hydroxide followed by washing with chloroform (100 ml×2). The pH value of this aqueous solution was adjusted to 7.4 with 1 N hydrochloric acid followed by extraction with chloroform (200 ml×3). The combined organic layer was dried over anhydrous sodium sulfate and filtered. The filtrate was concentrated under reduced pressure and the residue was recrystallized from ethanol and dried under reduced pressure to give 170 mg (42.6%) of the title compound as a yellow powder.

Melting point: 211-213° C. (decomp.).

¹H—NMR (400 MHz, 0.1 N—NaOD) δ: 0.57-0.74 (4H, m), 1.12-1.27 (1H, m), 1.36-1.48 (1H, m), 2.24 (1H, dm, J=37.60 Hz), 3.46 (3H, s), 3.53 (1H, t, J=8.79 Hz), 3.69 (1H, dd, J=25.40, 12.21 Hz), 3,86-3.94 (2H, m), 4.10 (1H, ddm, J=42.48, 12.70 Hz), 5.00 (1H, dm, J=63.97 Hz), 5.49 (1H, brd. J=54.69 Hz), 8.19 (1H, d, J=3.91 Hz).

Elemental analysis data: as C₂₁H₂₃F₃N₄O₄ calcd.: C, 55.75; H, 5.12; N, 12.38 found: C, 55.78; H, 5.20; N, 12.28.

### EXAMPLE 4

### 10-[4-(R)-(1-Aminocyclopropyl)-3-(S)-fluoro-1-pyrrolidinyl]-9-fluoro-2,3-dihydro-3-(S)-methyl-7-oxo-7H-pyrido[1, 2,3-de][1,4]benzoxazine-6-carboxylic acid

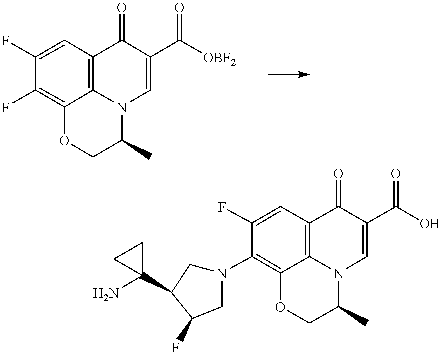

1-Benzyloxycarbonyl-4-(R)-(1-tert-butoxycarbonylaminocyclopropyl)-3-(S)-fluoropyrrolidine (913 mg, 2.41 mmol) was dissolved in methanol (50 ml). After adding 5% palladium-carbon catalyst (moisture content: 55.6%, 1.0 g), the mixture was stirred under a hydrogen atmosphere for 3 hours. After filtering off the catalyst through celite (washed with methanol), the filtrate was concentrated under reduced pressure. The residue thus obtained was dissolved in dimethyl sulfoxide (15 ml) and 9,10-difluoro-2,3-dihdyro-3-(S)-methyl-7-oxo-7H-pyrido[1, 2,3-de][1,4]benzoxazine-6-carboxylic acid-BF₂ chelate (661 mg, 2.01 mmol) and triethylamine (336 μl, 2.41 mmol) were added thereto. Then the mixture was stirred at room temperature for 3 days. After concentrating the liquid reaction mixture under reduced pressure, water was added to the residue. The yellow crystals thus precipitated were collected by filtration and washed with water. The obtained crystals were suspended in a solution (200 ml) of methanol:water=1:1. After adding triethylamine (4 ml), the mixture was heated under reflux for 4 hours. After allowing to cool, the liquid reaction mixture was concentrated under reduced pressure and the residue was dissolved in chloroform (200 ml) and washed with a 10% aqueous solution of citric acid (200 ml). The organic layer was dried over anhydrous sodium sulfate. After filtering, the filtrate was concentrated under reduced pressure. Under ice cooling, conc. hydrochloric acid (10 ml) was dropped into the residue followed by stirring at room temperature for 10 minutes. After adding 1 N hydrochloric acid (30 ml), the liquid reaction mixture was washed with chloroform (50 ml×2) and its pH value was adjusted to 12.0 with an aqueous solution of sodium hydroxide. The pH value of this aqueous solution was adjusted to 7.4 with 1 N hydrochloric acid followed by extraction with chloroform (500 ml×3). The combined organic layer was dried over anhydrous sodium sulfate and filtered. The filtrate was concentrated under reduced pressure and the residue was recrystallized from ethanol and dried under reduced pressure to give 459 mg (56.4%) of the title compound as pale yellow crystals.

Melting point: 230-231° C. (decomp.).

¹H—NMR (400 MHz, 0.1 N—NaOD) δ: 0.55-0.75 (4H, m), 1.52 (3H, d, J=6.84 Hz), 2.25 (1H, dm, J=36.62 Hz), 3.49 (1H, t, J=8.79 Hz), 3.70 (1H, dd, J=26.37, 11.72 Hz), 3.88 (1H, t, J=8.79 Hz), 4.10 (1H, dd, J=40.53, 12.70 Hz), 4.30 (1H, d, J=9.27 Hz), 4.50 (1H, d, J=9.28 Hz), 4.55-4.65 (1H, m), 5.47 (1H, dt, J=55.17, 3.42 Hz), 7.53 (1H, d, J=14.16 Hz), 8.33 (1H, s).

### Referential Example 3-1

### Ethyl 1-acetylcyclopropanecarboxylate

Ethyl acetoacetate (100 g, 0.77 mol) was dissolved in acetone (500 ml). To the obtained solution was added dibromoethane (361 g, 1.92 mol) and potassium carbonate (266 g, 1.92 mol) and the mixture was heated under reflux for 4 days. After filtering off the insoluble matters, the filtrate was distilled under reduced pressure (80° C./8 mmHg) to give 78.1 g (65.1%) of the title compound as a colorless oily substance.

¹H—NMR (400 MHz, CDCl₃) δ: 1.29 (3H, t, J=7.33 Hz), 1.47 (4H, s), 2.47 (3H, s), 4.21 (2H, q, J=7.33 Hz).

### Referential Example 3-2

### Ethyl 3-(1-ethoxycarbonylcyclopropyl)-2-fluoro-2-butenoate

To a solution (1500 ml) of ethyl 1-acetylcyclopropanecarboxylate (124.5 g, 0.797 mmol) in benzene was added zinc powder (156.4 g, 2.39 mmol). While heating under reflux, a catalytic amount of iodine was added thereto. Subsequently, a solution of ethyl bromofluoroacetate (94.23 ml, 0.797 mol) in benzene (200 ml) was dropped thereinto over 1 hour followed by heating under reflux for 1 hour. Under ice cooling, 1 N hydrochloric acid (1000 ml) was added to the liquid reaction mixture and the mixture was stirred for 1 hour. The organic layer taken up by phase separation was washed successively with 1 N hydrochloric acid, water and a saturated aqueous solution of sodium chloride, dried over anhydrous sodium sulfate and filtered. The filtrate was concentrated under reduced pressure and the residue was dissolved in pyridine (387 ml, 4.78 mol). After adding thionyl chloride (69.8 ml, 0.957 mol) at −10° C., the resultant mixture was stirred under ice cooling for 3 hours. Under ice cooling, the liquid reaction mixture was poured into 1 N hydrochloric acid (2000 ml) and ethyl acetate (1500 ml) was added thereto. The organic layer taken up by phase separation was washed successively with 1 N hydrochloric acid, water and a saturated aqueous solution of sodium chloride, dried over anhydrous sodium sulfate and filtered. The filtrate was concentrated under reduced pressure and the residue was dissolved in dichloromethane (500 ml). Under ice cooling, 1,8-azabicyclo[5.4.0]-7-undecene (131 ml, 0.877 mol) was dropped thereinto and then the resultant mixture was stirred at room temperature for 17 hours. After adding 1 N hydrochloric acid (2000 ml) and chloroform (1000 ml), the organic layer taken up by phase separation was washed with a saturated aqueous solution of sodium chloride, dried over anhydrous sodium sulfate and filtered. The filtrate was concentrated under reduced pressure and the residue thus obtained was subjected to flash silica gel column chromatography (eluent: n-hexane:ethyl acetate 4:1) 152.78 g (78.5%) of the title compound as an oily substance. The compound thus obtained, which was a mixture of geometrical isomers (about 1:1), was not separated but employed in the subsequent reaction as such.

### Referential Example 3-3

### (E)-Ethyl 4-bromo-3-(1-ethoxycarbonylcyclopropyl)-2-fluoro-2-butenoate

To a solution of ethyl 3-(1-ethoxycarbonyl-cyclopropyl)-2-fluoro-2-butenoate (152.78 g, 0.625 mol) in chloroform (1500 ml) were added N-bromosuccinimide (111.33 g, 0.625 mol) and a catalytic amount of 2, 2′-azobis-(isobutyronitrile) and then the resultant mixture was heated under reflux for 16 hours. Then the liquid reaction mixture was cooled and concentrated under reduced pressure. After adding benzene (300 ml), the insoluble matters were filtered off and the filtrate was concentrated under reduced pressure. The residue was subjected to flash silica gel column chromatography (eluent: n-hexane:ethyl acetate=4:1) to give 100.5 g (49.7%) of the title compound as a yellow oily substance. On the other hand, 75 g (37.1%) of (Z)-ethyl 4-bromo-3-(1-ethoxycarbonylcyclopropyl)-2-fluoro-2-butenoate (the geometrical isomer of the title compound) was obtained as a yellow oily substance with the use of another eluent (n-hexane:ethyl acetate=2:1).

(E)-isomer

¹H—NMR (400 MHz, CDCl₃) δ: 1.23 (3H, t, J=7.08 Hz), 1.38 (3H, t, J=7.08 Hz), 1.52-1.62 (4H, br), 4.11 (2H, q, J=7.08 Hz), 4.35 (2H, q, J=7.08 Hz), 4.54 (2H, s).

(Z)-isomer:

¹H—NMR (400 MHz, CDCl₃) δ: 1.21 (3H, t, J=7.08 Hz), 1.32 (3H, t, J=7.08 Hz), 1.52-1.62 (4H, br), 4.11 (2H, q, J=7.08 Hz), 4.13 (2H, s), 4.29 (2H, q, J=7.08 Hz).

### Referential Example 3-4

### 4-(1-Ethoxycarbonylcyclopropyl)-3-fluoro-1-[1-(S)-phenylethyl1-3-pyrrolin-2-one

To a solution of (E)-ethyl 4-bromo-3-(1-ethoxycarbonylcyclopropyl)-2-fluoro-2-butenoate (143 mmol) in ethanol (1000 ml) was added sodium hydrogencarbonate (30.08 g, 358 mmol). After dropping 1-(S)-phenylethylamine (20.31 ml, 158 mmol) thereinto at room temperature, the mixture was heated under reflux for 3 hours. Then the liquid reaction mixture was cooled and filtered through celite. The filtrate was concentrated under reduced pressure and the residue was subjected to flash silica gel column chromatography (eluent: n-hexane:ethyl acetate=2:1) to give 36.95 g (81.2%) of the title compound as an oily substance.

¹H—NMR (400 MHz, CDCl₃) δ: 1.16 (3H, t, J 7.08 Hz), 1.22-1.30 (2H, m), 1.55-1.59 (2H, m), 1.62 (3H, d, J=7.33 Hz), 3.76 (2H, ddd, J=128.42, 18.07, 5.37 Hz), 4.08 (2H, q, J=7.08 Hz), 5.56 (1H, q, J=7.33 Hz).

### Referential Example 3-5

### 4-(S)-(1-Ethoxycarbonylcycloproiyl)-3-(S)-fluoro-1-[1-(S)-phenylethyl]-2-pyrrolidone

To a solution of 4-(1-ethoxycarbonylcyclopropyl)-3-fluoro-1-[1-(S)-phenylethyl]-3-pyrrolin-2-one (587 mg, 1.85 mmol) in ethanol (5 ml) was added Raney nickel (R-100, 2 ml). Under a hydrogen atmosphere of 5 kg/cm², the mixture was stirred at room temperature for 1 hour. Next, Raney nickel (R-100, 3 ml) was further added and stirring was continued under the same conditions for 2.5 hours. After eliminating the catalyst by filtering through celite (washed with ethanol), the filtrate was concentrated under reduced pressure. The residue was subjected to flash silica gel column chromatography (eluent: n-hexane:ethyl acetate=3:1) to give 382 mg (64.6%) of the title compound as a colorless oily substance. The ¹H—NMR data of this compound agreed with the data of the compound obtained in Referential Example 2-2.

¹H—NMR (400 MHz, CDCl₃) δ: 0.57-0.63 (1H, m), 0.78-0.84 (1H, m), 1.07-1.13 (1H, m), 1.26 (3H, t, J=7.09 Hz), 1.23-1.29 (1H, m), 1.54 (3H, d, J=7.32 Hz), 2.59 (1H, t, J=8.30 Hz), 3.05 (1H, dq, J=28.81, 8.30 Hz), 3.25 (1H, t, J=8.30 Hz), 4.00-4.16 (2H, m), 5.15 (1H, dd, J=52.73, 6.35 Hz), 5.53 (1H, q, J=7.32 Hz), 7.27-7.38 (5H, m).

### Referential Example 3-6

### 4-(S)-(1-Carboxycyclopropyl)-3-(S)-fluoro-1-[1-(S)-phenylethyl]-2-pyrrolidone

4-(S)-(1-ethoxycarbonylcyclopropyl)-3-(S)-fluoro-1-[1-(S)-phenylethyl]-2-pyrrol-idone (12.56 g, 39.33 mmol) was dissolved in ethanol (120 ml) and a 1 N aqueous solution of sodium hydroxide (120 ml) was dropped thereinto. After stirring at 40° C. for 6 hours, ethanol was evaporated under reduced pressure. The residue was washed with chloroform (100 ml×2). Under ice cooling, the separated aqueous layer was acidified by dropping 1 N hydrochloric acid thereinto and then extracted successively with chloroform (300 ml×2) and diethyl ether (300 ml). The combined organic layer was dried over anhydrous sodium sulfate and filtered. The filtrate was concentrated under reduced pressure to give 10.24 g (89.4%) of the title compound as colorless needles.

¹H—NMR (400 MHz, CDCl₃) δ: 0.65-0.75 (1H, m), 0.85-0.95 (1H, m), 1.15-1.25 (1H, m), 1.26-1.36 (1H, m), 1.54 (3H, d, J=7.32 Hz), 2.60 (1H, t, J 7.8 Hz), 3.01 (1H, dq, J=27.83, 7.81 Hz), 3.28 (1H, t, J=7.81 Hz), 5.16 (1H, dd, J=52.74, 6.35 Hz), 5.53 (1H, q, Ji 7.32 Hz), 7.27-7.38 (5H, m).

### Referential Example 3-7

### 4-(R)-(1-tert-Butoxycarbonylaminocycloproyl )-3-(S1-fluoro-1-[1-(S)-phenylethyl]-2-pyrrolidone

§ Process by Hoffman rearrangement

To a solution of 4-(S)-(1-carboxycyclopropyl)-3-(S)-fluoro-1-[1-(S)-phenylethyl]-2-pyrrolidone (11.90 g, 40.85 mmol) in acetonitrile (160 ml) was added 1, 1′-carbonylidimidazole (13.25 g, 81.70 ml). The obtained mixture was stirred at room temperature for 30 minutes and then at 40° C. for additional 30 minutes. After cooling the liquid reaction mixture to room temperature, ammonia gas was bubbled thereinto for 30 minutes. After distilling off the solvent, chloroform (500 ml) was added to the residue followed by washing with water. The organic layer was dried over anhydrous sodium sulfate and filtered. The filtrate was concentrated under reduced pressure and the residue thus obtained was dissolved in tert-butyl alcohol (200 ml) and heated to 70° C. Then lead tetraacetate (purity 90% or more, 24.15 g, 49.02 mmol) was added thereto and the mixture was heated under reflux for 20 minutes. After cooling, sodium hydrogencarbonate was added followed by dilution with ethyl acetate (300 ml). Then the insoluble matters were filtered off and the filtrate was washed with a saturated aqueous solution of sodium bicarbonate. The organic layer was dried over anhydrous sodium sulfate and the solvent was evaporated under reduced pressure to give 10.52 g (71.7%) of the title compound.

§ Process by Curtius rearrangement

Under a nitrogen gas stream, toluene (100 ml) was added to 4-(S)-(1-carboxycyclopropyl)-3-(S)-fluoro-1-[1-(S)-phenylethyl]-2-pyrrolidone (3.66 g, 12.56 mmol). Next, triethylamine (3.50 ml, 25.13 mmol) was dropped thereinto at room temperature. When the liquid reaction mixture became a homogeneous system, diphenylphosphoric acid azide (2.71 ml, 12.56 mmol) was added and the resultant mixture was stirred at room temperature for 1 hour and then heated under reflux for 2 hours. Then tert-butyl alcohol (100 ml) was added to the liquid reaction mixture and the mixture was further heated under reflux for 21 hours. The liquid reaction mixture was cooled and concentrated under reduced pressure. The residue thus obtained was subjected to flash silica gel column chromatography (eluent: n-hexane:ethyl acetate=1:1) to give 3.30 g (72.5%) of the title compound as a colorless oily substance.

¹H—NMR (400 MHz, CDCl₃) δ: 0.58-0.66 (1H, m), 0.70-0.82 (2H, m), 0.88-0.96 (1H, m), 1.31 (9H, s), 1.54 (3H, d, J=7.33 Hz), 2.36-2.52 (1H, m), 2.86 (1H, t, J=8.30 Hz), 3.32 (1H, t, J=8.30 Hz), 4.99 (1H, dd, J=52.73, 6.35 Hz), 4.99 (1H, s), 5.46 (1H, q, J=7.33 Hz), 7.27-7.42 (5H, m).

### Referential Example 3-8

### 4-(R)-(1-tert-Butoxycarbonylaminocyclopropyl)-3-(S)-fluoro-1-[1-(S)-phenylethyl]pyrrolidine

Under a nitrogen atmosphere, a 1 M solution of borane-tetrahydrofuran complex in tetrahydrofuran (120 ml) was dropped under ice cooling into a solution of 4-(R)-(1-tert-butoxycarbonylaminocyclopropyl)-3-(S)-fluoro-1-1-(S)-phenylethyl]-2-pyrrolidone in tetrahydrofuran (120 ml) and the mixture was stirred at room temperature for 5 hours. After evaporating the solvent under reduced pressure, a solvent mixture (200 ml) of ethanol with water (4:1) and triethylamine (20 ml) were added to the residue followed by heating under reflux for 2 hours. Then the liquid reaction mixture was concentrated under reduced pressure and chloroform (400 ml) was added to the residue. After washing with a saturated aqueous solution of sodium chloride, the organic layer was dried over anhydrous sodium sulfate and filtered. The filtrate was concentrated under reduced pressure and the residue was subjected to flash silica gel column chromatography (eluent: n-hexane:ethyl acetate=1:2) to give 7.84 g (99.4%) of the title compound as a colorless oily substance.

¹H—NMR (400 MHz, CDCl₃) δ: 0.54-0.62 (1H, m), 0.70-0.95 (3H, m), 1.35 (3H, d, J=6.35 Hz), 1.42 (9H, s), 2.27-2.45 (2H, m), 2.46-2.56 (1H, m), 2.60-2.75 (1H, m), 3.00-3.15 (1H, m), 3.29 (1H, q, J=6.35 Hz), 5.06 (1H, s), 5.05-5.20 (1H, m), 7.20-7.32 (5H, m).

### EXAMPLE 5

### 7-[4-(R)-(1-Aminocyclopropyl)-3-(S)-fluoro-1-pyrrolidinyl]-6-fluoro-1-[2-(S)-fluoro-1-(R)-cycloproyyl]-1,4-dihydro-8-methoxy-4-oxoquinoline-3-carboxylic acid

4-(R)-(1-tert-butoxycarbonylaminocyclopropyl)-3-(S)-fluoro-1-[1-(S)-phenylethyl]pyrrolidine (6.32 g, 18.14 mmol) was dissolved in ethanol (150 ml). After adding 10% palladium-carbon catalyst (moisture content: 50.2%, 6.0 g), the mixture was stirred at 40° C. under a hydrogen atmosphere for 36 hours. After filtering off the catalyst through celite (washed with ethanol), the filtrate was concentrated under reduced pressure. The residue thus obtained was dissolved in dimethyl sulfoxide (20 ml) and 6,7-difluoro-1-[2-(S)-fluoro-1-(R)-cyclopropyl]-1,4-dihydro-8-methoxy-4-oxoquinoline-3-carboxylic acid-BF₂ chelate (4.37 g, 12.09 mmol) and triethylamine (5.05 ml, 36.23 mmol) were added thereto. Then the mixture was stirred at room temperature for 23 hours. After concentrating the liquid reaction mixture under reduced pressure, water was added to the residue. The solid matter thus precipitated was collected by filtration and washed with water. The obtained solid was suspended in a solution (400 ml) of methanol:water=10:1. After adding triethylamine (20 ml), the mixture was heated under reflux for 4 hours. After allowing to cool, the liquid reaction mixture was concentrated under reduced pressure and the residue was dissolved in chloroform (500 ml) and washed with a 10% aqueous solution of citric acid (500 ml). The organic layer was dried over anhydrous sodium sulfate. After filtering, the filtrate was concentrated under reduced pressure. Under ice cooling, conc. hydrochloric acid (30 ml) was dropped into the residue followed by stirring at room temperature for 2 hours. After adding 1 N hydrochloric acid (30 ml), the liquid reaction mixture was washed with chloroform (100 ml×2) and its pH value was adjusted to 12.0 with an aqueous solution of sodium hydroxide. The pH value of this aqueous solution was adjusted to 7.4 with 1 N hydrochloric acid followed by extraction with chloroform (500 ml×4). The combined organic layer was dried over anhydrous sodium sulfate and filtered. The filtrate was concentrated under reduced pressure and the residue was recrystallized from ethanol and dried under reduced pressure to give 4.09 g (77.3%) of the title compound as pale yellow crystals.

Melting point: 218.5-219.8° C. (decomp.).

¹H—NMR (400 MHz, 0.1 N—NaOD) δ: 0.57-0.74 (4H, m), 1.32-1.45 (1H, m), 1.48-1.60 (1H, m), 2.20-2.38 (1H, m), 3.53-3.58 (1H, m), 3.58 (3H, s), 3.72 (1H, dd, J=25.88, 13.19 Hz), 3.86-3.93 (1H, m), 4.00-4.18 (2H, m), 5.50 (1H, dm, J=63.96 Hz), 5.51 (lh, brd. J=54.68 Hz), 7.68 (1H, d, 14.16 Hz), 8.19 (1H, d, J=3.91 Hz).

Elemental analysis data: as C₂₁H₂₂F₃N₃O₄ calcd.: C, 57.66; H, 5.07; N, 9.61 found: C, 57.52; H. 5.02; N. 9.48.

### EXAMPLE 6

### 10-[4-(R)-(1-Aminocyclopropyl)-3-(S)-fluoro-1-pyrrolidinyl]-9-fluoro-2,3-dihydro-3-(S)-methyl-7-oxo-7H-pyrido[1,2, 3-de][1,4]benzoxazine-6-carboxylic acid hydrochloride

4-(R)-(1-tert-butoxycarbonylaminocyclopropyl)-3-(S)-fluoro-1-[1-(S)-phenylethyl]pyrrolidine (1.12 g, 3.21 mmol) was dissolved in ethanol (20 ml). After adding 10% palladium-carbon catalyst (moisture content: 50.2%, 1.12 g), the mixture was stirred at 40° C. under a hydrogen atmosphere for 4 hours. After filtering off the catalyst through celite (washed with ethanol), the filtrate was concentrated under reduced pressure. The residue thus obtained was dissolved in dimethyl sulfoxide (10 ml) and 9.10-difluoro-2,3-dihydro-3-(S)-methyl-7-oxo-7H-pyrido[1,2,3-de][1,4]benzoxazine-6-carboxylic acid-BF₂ chelate (705 mg, 2.14 mmol) and triethylamine (0.60 ml, 4.29 mmol) were added thereto. Then the mixture was stirred at room temperature for 3 hours. After concentrating the liquid reaction mixture under reduced pressure, water was added to the residue. The yellow crystals thus precipitated were collected by filtration and washed with water. The obtained crystals were suspended in methanol (moisture content: 10%, 100 ml). After adding triethylamine (5 ml), the mixture was heated under reflux for 14 hours. After allowing to cool, the liquid reaction mixture was concentrated under reduced pressure and the residue was dissolved in chloroform (200 ml) and washed with a 10% aqueous solution of citric acid (200 ml). The organic layer was dried over anhydrous sodium sulfate. After filtering, the filtrate was concentrated under reduced pressure. Under ice cooling, conc. hydrochloric acid (10 ml) was dropped into the residue followed by stirring at room temperature for 10 minutes. After adding 1 N hydrochloric acid (30 ml), the liquid reaction mixture was washed with chloroform (50 ml×2) and its pH value was adjusted to 12.0 with an aqueous solution of sodium hydroxide. The pH value of this aqueous solution was adjusted to 7.4 with 1 N hydrochloric acid followed by extraction with chloroform (500 ml×3). The combined organic layer was dried over anhydrous sodium sulfate and filtered. The filtrate was concentrated under reduced pressure and 1 N hydrochloric acid (5.0 ml) was dropped into the residue under ice cooling. After stirring at the same temperature for 5 minutes, the liquid reaction mixture was concentrated under reduced pressure (azeotropic distillation with ethanol, thrice). The residue was recrystallized from ethanol and dried under reduced pressure to give 685 mg (68.9%) of the title compound as a pale yellow powder.

Melting point: 197-199° C. (decomp.).

¹H—NMR (400 MHz, 0.1 N—NaOD) δ: 0.59-0.68 (4H, m), 1.52 (3H, d, J=6.84 Hz), 2.39 (1H, dt, J=29.30, 7.81 Hz), 3.37 (1H, t, J=7.81 Hz), 3.74-3.90 (3H, m), 3.95 (1H, t, J=9.76 Hz), 4.36 (1H, d, J=10.26 Hz), 4.53 (1H, d, J=11.23 Hz), 4.62 (1H, q, J=6.84 Hz), 5.34 (1H, brd, J=54.02 Hz), 7.57 (1H, d, J=13.67 Hz), 8.35 (1H, s).

Elemental analysis data: as C₂₀H₂₁F₂N₃O₄.HCl.1.25H₂O calcd.: C, 51.73; H, 5.32; N, 9.05 found: C, 51.97; H, 5.34; N, 9.10.

### EXAMPLE 7

### 5-Amino-7-[4-(R)-(1-aminocyclopropyl)-3-(S)-fluoro-1-pyrrolidinyl]-6-fluoro-1-[2-(S)-fluoro-1-(R)-cyclopropyl]-1,4-dihydro-8-methyl-4-oxoquinoline-3-carboxylic acid hydrochloride

4-(R)-(1-tert-butoxycarbonylaminocyclopropyl)-3-(S)-fluoro-1-[1-(S)-phenylethyl]pyrrolidine (2.11 g, 6.06 mmol) was dissolved in ethanol (40 ml). After adding 10% palladium-carbon catalyst (moisture content: 50.2%, 2.11 g), the mixture was stirred under a hydrogen atmosphere for 5 hours. After filtering off the catalyst through celite (washed with ethanol), the filtrate was concentrated under reduced pressure. The residue thus obtained was dissolved in dimethyl sulfoxide (6 ml) and 5-amino-6,7-difluoro-1-[2-(S)-fluoro-1-(R)-cyclopropyl]-1,4-dihydro-8-methyl-4-oxoquinoline-3-carboxylic acid (1.26 g, 4.04 mmol) and triethylamine (14 ml) were added thereto. Then the mixture was stirred in a nitrogen atmosphere in an oil bath at 150° C. for 8 days. After allowing to cool, dimethyl sulfoxide was evaporated under reduced pressure and the residue was dissolved in chloroform (80 ml) and washed with a 10% aqueous solution of citric acid (80 ml). The organic layer was dried over anhydrous sodium sulfate. After filtering, the filtrate was concentrated under reduced pressure. Under ice cooling, conc. hydrochloric acid (10 ml) was dropped into the residue followed by stirring at room temperature for 30 minutes. After adding 1 N hydrochloric acid (30 ml), the liquid reaction mixture was washed with chloroform (50 ml×2) and its pH value was adjusted to 12.0 with an aqueous solution of sodium hydroxide. The pH value of this aqueous solution was adjusted to 7.4 with 1 N hydrochloric acid followed by extraction with chloroform (500 ml×3). The combined organic layer was dried over anhydrous sodium sulfate and filtered. The filtrate was concentrated under reduced pressure and 1 N hydrochloric acid (5.0 ml) was dropped into the residue under ice cooling. After stirring at the same temperature for 5 minutes, the liquid reaction mixture was concentrated under reduced pressure (azeotropic distillation with ethanol, thrice). The residue was recrystallized from ethanol and dried under reduced pressure to give 561 mg (28.8%) of the title compound as a pale yellow powder.

¹H—NMR (400 MHz, 0.1 N—NaOD) δ: 0.55-0.71 (4H, m), 1.10-1.21 (1H, m), 1.46-1.58 (1H, m), 2.30 (3H, s), 2.21-2.35 (1H, m), 3.32 (1H, t, J 8.79 Hz), 3.49 (1H, dd, J=25.88, 12.21 Hz), 3.85-3.97 (2H, m), 4.11 (1H, ddm, J=40.77, 12.45 Hz), 4.97 (1H, dm, J=70.31 Hz), 5.49 (1H, brd, J=55.18 Hz), 8.27 (1H, d, J=3.42 Hz).

Elemental analysis data: as C₂₁H₂₃F₃N₄O₃•HCl,•0.5H₂O calcd.: C, 52.34; H, 5.23; N, 11.63 found: C, 52.32; H, 5.36; N, 11.76.

### EXAMPLE 8

### 8-Amino-10-[4-(R)-(1-aminocyclopropyl)-3-(S)-fluoro-1-pyrrolidinyl]-9-fluoro-2,3-dihydro-3-(S)-methyl-7-oxo-7H-pyrido[1,2,3-de][1,4]benzoxazine-6-carboxylic acid

4-(R)-(1-tert-Butoxycarbonylaminocyclopropyl)-3-(S)-fluoro-1-[1-(S)-phenylethyl]pyrrolidine (900 mg, 2.58 mmol) was dissolved in ethanol (20 ml). After adding 10% palladium-carbon catalyst (moisture content: 50.2%, 900 mg), the mixture was stirred under a hydrogen atmosphere for 4 hours. After filtering off the catalyst through celite (washed with ethanol), the filtrate was concentrated under reduced pressure. The residue thus obtained was dissolved in dimethyl sulfoxide (20 ml) and 8-amino-9,10-difluoro-2, 3-dihydro-3-(S)-methyl-7-oxo-7H-pyrido[1,2, 3-de][1,4]benzoxazine-6-carboxylic acid (593 mg, 2.00 mmol) and triethylamine (3 ml) were added thereto. Then the mixture was stirred in a nitrogen atmosphere in an oil bath at 100° C. for 25 hours. After allowing to cool, dimethyl sulfoxide was evaporated under reduced pressure and the residue was dissolved in chloroform (100 ml) and washed with a 10% aqueous solution of citric acid (80 ml). The organic layer was dried over anhydrous sodium sulfate. After filtering, the filtrate was concentrated under reduced pressure. Under ice cooling, conc. hydrochloric acid (10 ml) was dropped into the residue followed by stirring at room temperature for 30 minutes. After adding 1 N hydrochloric acid (30 ml), the liquid reaction mixture was washed with chlorofor (50 ml×4) and its pH value was adjusted to 12.0 with an aqueous solution of sodium hydroxide. The pH value of this aqueous solution was adjusted to 7.4 with 1 N hydrochloric acid followed by extraction with chloroform (500 ml×3). The combined organic layer was dried over anhydrous sodium sulfate and filtered. The filtrate was concentrated under reduced pressure and the residue was recrystallized from ethanol and dried under reduced pressure to give 640 mg (76.0%) of the title compound as a yellow powder.

Melting point: 247-250° C. (decomp.).

¹H—NMR (400 MHz, 0.1 N—NaOD) δ: 0.53-0.68 (4H, m), 1.45 (3H, d, J=6.98 Hz), 2.18 (1H, dt, J=36.14, 7.81 Hz), 3.38 (1H, t, J=7.81 Hz), 3.66 (1H, dd, J=25.63, 12.94 Hz), 3.82 (1H, t, J=10.01 Hz), 3.99-4.12 (3H, m), 4.32 (1H, d, J=11.24 Hz), 4.44 (1H, d, J=6.98 Hz), 5.43 (1H, d, J=54.69 Hz), 8.13 (1H, s).

Elemental analysis data: as C₂₀H₂₂F₂N404 calcd.: C, 57.14; H, 5.27; N. 13.33 found: C, 56.86; H, 5.26; N, 13.39.

### Referential Example 4-1

### Ethyl 1-acetylcyclobutanecarboxylate

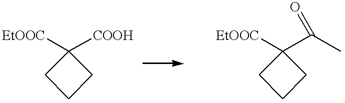

Ethyl hydrogen 1,1-cyclobutanecarboxylate (64.43 g, 374 mmol) was dissolved in methylene chloride (500 ml). Under ice cooling, oxalyl chloride (65.29 ml, 748 mmol) was added thereto followed by the addition of a catalytic amount of N,N-dimethylformamide. Then the resultant mixture was stirred at room temperature for 1.5 hours. After evaporating the solvent, the residue was subjected to azeotropic distillation together with toluene twice to give an acid chloride.

On the other hand, copper (I) iodide (85.52 g, 449 mmol) was suspended in tetrahydrofuran (1 1) under a nitrogen gas stream. At −20° C., a 1.4 M solution (294 ml) of methyllithium in diethyl ether was dropped thereinto and the mixture was stirred at the same temperature for 1 hour. Subsequently, the above-mentioned acid chloride was dissolved in tetrahydrofuran (300 ml) and dropped thereinto at the same temperature followed by stirring for 1.5 hours. After the completion of the reaction, the reaction temperature was brought back to room temperature and a 10% aqueous solution of citric acid (500 ml) was added to the mixture. After evaporating tetrahydrofuran, ethyl acetate (1 1) was added to the residue. Then the insoluble matters were filtered off and the residue was washed successively with a 5% aqueous solution of sodium thiosulfate (300 ml) and a saturated aqueous solution of sodium chloride (300 ml) and dried over anhydrous sodium sulfate. After evaporating the solvent, the obtained residue was subjected to silica gel column chromatography (eluent: n-hexane:ethyl acetate=4:1) to give 56.70 g (89%) of the title compound as an oily substance.

¹H—NMR (400 MHz, CDCl₃) δ: 1.27 (3H, t, J=7.33 Hz), 1.82-2.01 (2H, m), 2.12 (3H, s), 2.45-2.55 (4H, m), 4.20-4.24 (2H, m).

### Referential Example 4-2

### Ethyl 1-ethoxycarbonyl-β-hydroxy-β-methyl-cyclobutylpropanoate

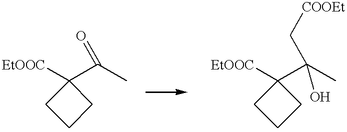

Ethyl 1-acetylcyclobutanecarboxylate (13.79 g, 81 minol) was dissolved in tetrahydrofuran (50 ml) and zinc powder (10.59 g) and a catalytic amount of iodine were added thereto. While heating under reflux, a solution (100 ml) of ethyl bromoacetate (13.48 ml, 121 mmnol) in tetrahydrofuran was dropped thereinto. Then the liquid reaction mixture was heated under reflux for additional 1 hour and allowed to cool. After adding 1 N hydrochloric acid (100 ml), the solvent was evaporated and ethyl acetate (500 ml) was added. The insoluble matters were filtered off, washed with a saturated aqueous solution of sodium chloride (300 ml) and dried over anhydrous sodium sulfate. After evaporating the solvent, the title compound was obtained in a quantitative amount as an oily substance.

¹H—NMR (400 MHz, CDCl₃) δ: 1.24-1.32 (9H, m), 1.73-1.87 (2H, m), 2,21-2.34 (2H,1m), 2.41-2.57 (5H, m), 4.16-4.21 (4H, m).

### Referential Example 4-3

### (E)-Ethyl 3-(1-ethoxycarbonylcyclobutyl)-2-butenoate

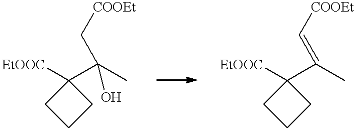

Ethyl 1-ethoxycarbonyl-β-hydroxy-β-methyl-cyclobutylpropanoate (22.27 g, 86 mmol) was dissolved in pyridine (42 ml) and thionyl chloride (8.18 ml, 112 mmol) was dropped thereinto at −10° C. After the completion of the reaction, the liquid reaction mixture was poured into ice water (250 ml) and extracted with ethyl acetate (100 ml×3). The combined organic layer was washed with 1 N hydrochloric acid (100 ml) and a saturated aqueous solution of sodium chloride (100 ml) and dried over anhydrous sodium sulfate. After evaporating the solvent, the residue thus obtained was dissolved in methylene chloride (250 ml). Then 1,8-diazabicyclo[5,4,0]-7-undecene (12.89 ml) was dropped thereinto at 0° C. and the obtained mixture was stirred at room temperature for 18 hours. After the completion of the reaction, the solvent was evaporated. Ice water (100 ml) was added to the residue followed by extraction with ethyl acetate (200 ml×3). The combined organic layer was washed with 1 N hydrochloric acid (100 ml) and a saturated aqueous solution of sodium chloride (100 ml) and dried over anhydrous sodium sulfate. After evaporating the solvent, the residue was subjected to silica gel column chromatography (eluent: n-hexane:ethyl acetate=4:1) to give 16.91 g (82%) of the title compound as an oily substance.

¹H—NMR (400 MHz, CDCl₃) δ: 1.24 (3H, t, J 6.83 Hz), 1.29 (3H, t, J=7.32 Hz), 1.74-1.80 (2H, m), 1.94-2.04 (1H, m), 2.07 (3H, d, J=1.47 Hz), 2.12-2.30 (2H, m), 2.12-2.30 (2H, m), 2.50-2.57 (2H, m), 4.13-4.20 (4H, m).

### Referential Example 4-4

### 4-(1-Ethoxycarbonylcyclobutyl)-1-[1-(S)-phenylethyl]-3-pyrrolin-2-one

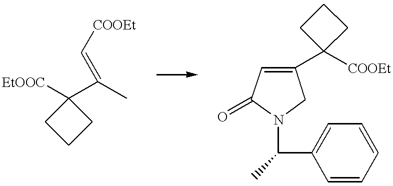

(E)-Ethyl 3-(1-ethoxycarbonylcyclobutyl)-2-butenoate (16.91 g, 70 mmol) was dissolved in chloroform (180 ml) and N-bromosuccinimide (12.53 g, 70 mmol) and a catalytic amount of azobisisobutyronitrile were added thereto. The obtained mixture was heated under reflux for 18 hours. After the completion of the reaction, the solvent was evaporated and carbon tetrachloride (100 ml) was added to the residue. Then the insoluble matters were filtered off and the filtrate was concentrated. The residue was dissolved in ethanol (100 ml) and sodium hydrogencarbonate (11.82 g, 140 mmol) was added thereto. Next, (S)-phenylethylamine (9.87 ml, 77 mmol) was dropped thereinto at room temperature. After the completion of the addition, the resultant mixture was heated under reflux for 3 hours. After the completion of the reaction, the solvent was evaporated and methylene chloride (300 ml) was added to the residue. The insoluble matters were filtered off and the solvent was evaporated. The residue thus obtained was subjected to silica gel column chromatography (eluent: n-hexane:ethyl acetate=1:1) to give 19.57 g (43%) of the title compound as an oily substance.

¹H—NMR (400 MHz, CDCl₃) δ: 1.17 (3H, t, J=7.33 Hz), 1.74-1.80 (2H, m), 1.59 (3H, d,J=6.84 Hz), 1.84-2.01 (2H, m), 2.15-2.28 (2H, m), 2.60-2.69 (2H, m), 3.56 (2H, d, J=9.04 Hz), 3.88 (2H, d, J=9.04 Hz), 4.13 (2H, q, J=7.32 Hz), 5.50-5.59 (1H, m), 6.03 (2H, s), 7.26-7.35 (5H,

### Referential Example 4-5

### 4-(1-Ethoxycarbonylcyclobutyl)-1-[(S)-phenylethyl]-2-pyrrolidone

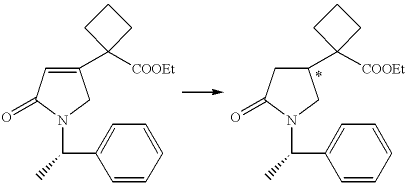

4-(1-Ethoxycarbonylcyclobutyl)-1-[(S)-phenylethyl]-3-pyrrolin-2-one (9.57 g, 31 mmol) was dissolved in ethanol (150 ml) and platinum oxide (230 mg) was added thereto. The obtained mixture was stirred in a hydrogen atmosphere for 18 hours. After the completion of the reaction, the liquid reaction mixture was filtered and concentrated. The residue thus obtained was subjected to silica gel column chromatography thrice (eluent: n-hexane:ethyl acetate 1:1) to give 2.3 g (24%) of an optical isomer A of the title compound and 7.1 g (74%) of another optical isomer B thereof each as an oily substance.

Optical isomer A

¹H—NMR (400 MHz, CDCl₃) δ: 1.26 (3H, t, J=6.83 Hz), 1.49 (2H, d, J=7.32 Hz), 1.83-1.95 (4H, m), 2.38-2.54 (4H, m), 2.66-2.74 (1H, m), 3.01 (1H, t, 8.30 Hz), 3.14 (1H, d, J=5.86, 9.77 Hz), 4.09-4.18 (2H, m), 5.48 (1H, dd, J=7.32, 14.16 Hz), 7.27-7.35 (5H, m).

Optical isomer B

¹H—NMR (400 MHz, CDCl₃) δ: 1.17 (3H, t, J=7.32 Hz), 1.52 (2H, d, J=7.33 Hz), 1.68-1.92 (4H, m), 2.23-2.43 (3H, m), 2.50-2.57 (1H, m), 2.73-2.86 (2H, m), 3.37 (1H, t, J=8.30 Hz), 4.05 (2H, q, J=7.32 Hz), 5.50 (1H, dd, J=7.32, 14.16 Hz), 7.24-7.35 (5H, m).

### Referential Example 4-6

### trans 4-(1-Ethoxycarbonylcyclobutyl)-3-fluoro-1-[1-(S)-phenylethyl]-2-pyrrolidone (optical isomer B)

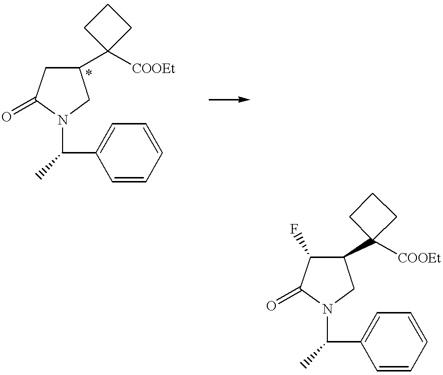

Under a nitrogen atmosphere, diisopropylamine (2.55 ml, 18.2 mmol) was dissolved in dry tetrahydrofuran (120 ml). After cooling the solution to −78° C., a 1.63 M solution of n-butyllithium in n-hexane (11.2 ml, 18.2 mmol) was dropped thereinto over 10 minutes. Then the liquid reaction mixture was stirred at 0° C. for 15 minutes and cooled to −78° C. Next, a solution (30 ml) of 4-(1-ethoxycarbonylcyclopropyl)-1-[1-(S)-phenylethyl]-2-pyrrolidone (optical isomer B; 4.42 g, 14.01 mmol) in dry tetrahydrofuran was dropped thereinto over 15 minutes. The liquid reaction mixture was stirred at −78° C. for 1 hour. Then a solution (25 ml) of N-fluorobenzenedisulfonimide (7.07 g, 22,42 mmol) in dry tetrahydrofuran was dropped thereinto over 5 minutes. The liquid reaction mixture was stirred at −78° C. for 30 minutes and then heated to room temperature followed by stirring for additional 20 minutes. Under ice cooling, a saturated aqueous solution of ammonium chloride (200 ml) was added to the liquid reaction mixture. After evaporating tetrahydrofuran, the aqueous layer was extracted with ethyl acetate (200 ml×2). The combined organic layer was washed with water (200 ml×3), dried over anhydrous sodium sulfate and filtered. The filtrate was concentrated under reduced pressure and the residue was subjected to silica gel column chromatography (eluent: n-hexane:ethyl acetate=1:1) to give 3.88 g (83%) of the title compound as an oily substance.

¹H—NMR (400 MHz, CDCl₃) δ: 1.14 (3H, t, J 6.83 Hz), 1.57 (2H, d, J 6.83 Hz), 1.88-2.08 (4H, m), 2.33-2.58 (3H, m), 2.81-2.92 (1H, m), 3.42 (1H, t, J=9.77 Hz), 3.93-4.07 (2H, m), 5.18 (1H, dd, J=6.83, 53.22 Hz), 5.51 (1H, dd, J=7.32, 14.16 Hz), 7.25-7.34 (5H, m).

### Referential Example 4-7

### cis 4-(1-Ethoxycarbonylcyclobutyl)-3-fluoro-1-[1-(S)-phenylethyl]-2-pyrrolidone (optical isomer B)

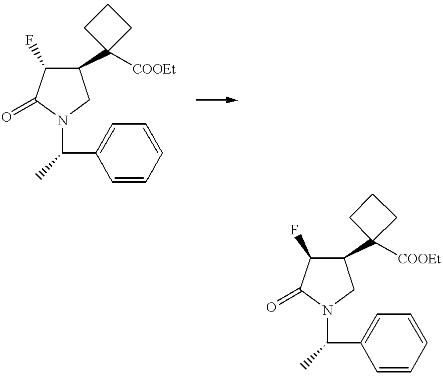

Under a nitrogen atmosphere, diisopropylamine (2.97 ml, 21.19 mmol) was dissolved in dry tetrahydrofuran (30 ml). After cooling the solution to −78 QC, a 1.63 M solution of n-butyllithium in n-hexane (10.8 ml, 17.60 mmol) was dropped thereinto over 5 minutes. Then the liquid reaction mixture was stirred at 0° C. for 15 minutes and cooled to −78° C. Next, a solution (30 ml) of trans 4-(1-ethoxycarbonyl-cyclopropyl)-3-fluoro-1-[1-(S)-phenylethyl]-2-pyrrolidone (optical isomer B; 4.71 g, 14.13 mmol) in dry tetrahydrofuran was dropped thereinto over 5 minutes. The liquid reaction mixture was stirred at −78° C. for 3 minutes. Then it was dropped into a solution (40 ml) of 2,6-di-tert-butylphenol (4.37 g, 21.18 mmol) in dry tetrahydrofuran over 5 minutes. The liquid reaction mixture was stirred at −78° C. for 10 minutes and a saturated aqueous solution of ammonium chloride (200 ml) was added thereto. Next, the liquid reaction mixture was brought back to room temperature and the organic layer was taken up. The aqueous layer was extracted with chloroform (100 ml×2). The combined organic layer was washed with water (100 ml×2), dried over anhydrous sodium sulfate and filtered. The filtrate was concentrated under reduced pressure and the residue was subjected to silica gel column chromatography to give 1.96 g (42%) of the starting compound (eluent: n-hexane:ethyl acetate=2:1) and 1.79 g (38%) of the title compound (eluent: n-hexane:ethyl acetate=3:2) each as an oily substance.

¹H—NMR (400 MHz, CDCl₃) δ: 1.22 (3H, t, J=6.83 Hz), 1.56-1.58 (3H, d, J=6.83 Hz), 1.84-2.42 (6H, m), 2.83-2.97 (1H, m), 3.15-3.24 (1H, m), 3.36-3.43 (1H, m), 4.11-4.17 (2H, m), 5.07 (1H, dd, J=-6.83, 52.24 Hz), 5.56 (1H, q, J=7.33 Hz), 7.26-7.36 (5H, m).

### Referential Example 4-8

### cis 4-(1-Carboxycyclobutyl)-3-fluoro-1-[1-(S)-phenylethyl]-2-pyrrolidone (optical isomer B)

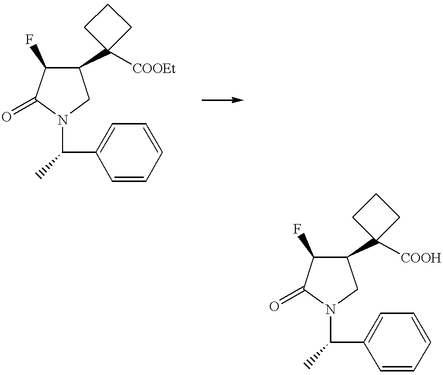

cis 4-(1-Ethoxycarbonylcyclobutyl)-3-fluoro-1-[1-(S)-phenylethyl)-2-pyrrolidone (optical isomer B; 1.79 g, 5.37 mmol) was dissolved in methanol (10 ml) and a 1 N aqueous solution of sodium hydroxide was dropped thereinto. The liquid reaction mixture was stirred at 40° C. for 18 hours and then methanol was evaporated under reduced pressure. Water (50 ml) was added to the residue followed by washing with chloroform (100 ml). The aqueous layer thus separated was acidified by dropping 1 N hydrochloric acid thereinto and then extracted with chloroform (100 ml×2). The combined organic layer was dried over anhydrous sodium sulfate and filtered. The filtrate was concentrated under reduced pressure to give the title compound in a quantitative amount as a crude product.

### Referential Example 4-9

### cis 4-(1-tert-Butoxycarbonylaminocyclobutyl)-3-fluoro-1-[1-(S)-phenylethyl]-2-pyrrolidone (optical isomer B)

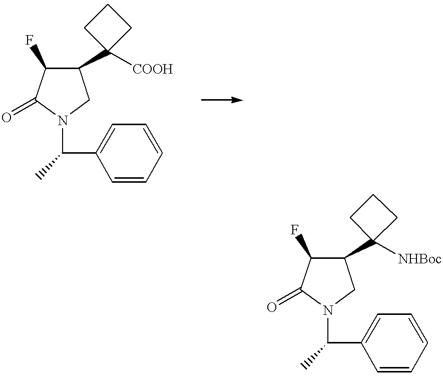

cis 4-(1-Carboxycyclobutyl)-3-fluoro-1-[1-(S)-phenylethyl]-2-pyrrolidone (optical isomer B; 1.92 g, 6.29 mmol) was dissolved in dry acetonitrile (30 ml) and N,N′-carbonyldiimidazole (1.33 g, 8.20 mmol) was added thereto. The liquid reaction mixture was stirred at 60° C. for 1 hour. Then ammonia was bubbled thereinto at room temperature for 10 minutes. After concentrating the liquid reaction mixture under reduced pressure, water (100 ml) was added to the residue followed by washing with chloroform (100 ml×2). The combined organic layer was dried over anhydrous sodium sulfate and filtered. The filtrate was concentrated under reduced pressure and the residue thus obtained was dissolved in tert-butyl alcohol (50 ml). After adding lead tetraacetate (6.32 g, 14.25 mmol), the mixture was heated under reflux for 1 hour. The liquid reaction mixture was then allowed to cool followed by the addition of diethyl ether (50 ml) and sodium hydrogencarbonate (6 g). Next, it was stirred at room temperature. for 10 minutes and filtered. The filtrate was concentrated under reduced pressure and ethyl acetate (100 ml) was added to the residue. The obtained mixture was washed with a saturated aqueous solution of sodium bicarbonate, dried over anhydrous sodium sulfate and filtered. The filtrate was concentrated under reduced pressure to give 1.74 g (65%) of the title compound as an oily substance.

¹H—NMR (400 MHz, CDCl₃) δ: 1.40 (9H, s), 1.92-2.21 (6H, m), 3.04-3.12 (1H, m), 3.31-3.38 (1H, m), 4.87 (1H, brs), 5.01 (1H, dd, J=5.86, 52.73 Hz), 5.52 (1H, dd, J=7.32, 14.16 Hz), 7.30-7.38 (5H, m).

### Referential Example 4-10

### cis 1-[1-(S)-phenylethyl]-4-(1-tert-butoxycarbonylaminocyclobutyl)-3-fluoropyrrolidone (optical isomer B)

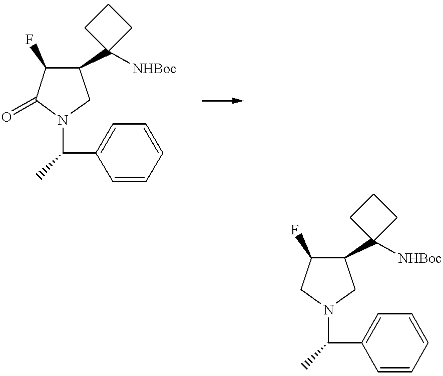

cis 4-(1-tert-Butoxycarbonylaminocyclobutyl)-3-fluoro-1-[1-(S)-phenylethyl]-2-pyrrolidone (optical isomer B; 1.74 g, 4.62 mmol) was dissolved in tetrahydrofuran (30 ml). At 0° C., borane-tetrahydrofuran complex salt (13.86 ml) was added thereto and the resultant mixture was stirred at room temperature for 2 days. After the completion of the reaction, the solvent was evaporated and water (50 ml) was added to the residue followed by extraction with chloroform (100 ml×2). The combined organic layer was dried over anhydrous sodium sulfate and filtered. The filtrate was concentrated under reduced pressure and the residue thus obtained was dissolved in 80% moisture-containing methanol (40 ml). After adding triethylamine (10 ml), the mixture was heated under reflux for 2 hours. After evaporating the solvent, the obtained residue was subjected to silica gel column chromatography (eluent: n-hexane:ethyl acetate=2:1) to give 1.13 g (67%) of the title compound as an oily substance.

¹H—NMR (400 MHz, CDCl₃) δ: 1.37 (3H, d, J 6.35 Hz), 1.44 (9H, s), 1.65-2.58 (7H, m), 2.70-2.92 (4H, m), 3.27-3.32 (1H, m), 5.14 (1H, brd), 5.53 (1H, brs), 7.22-7.33 (5H, m).

### EXAMPLE 9

### 5-Amino-7-[cis 4-(1-aminocyclobutyl)-3-fluoro-1-pyrrolidinyl]-6-fluoro-1-[2-(S)-fluoro-1-(R)-cyclopropyl]-1,4-dihydro-8-methyl-4-oxoguinoline-3-carboxylic acid (optical isomer B)

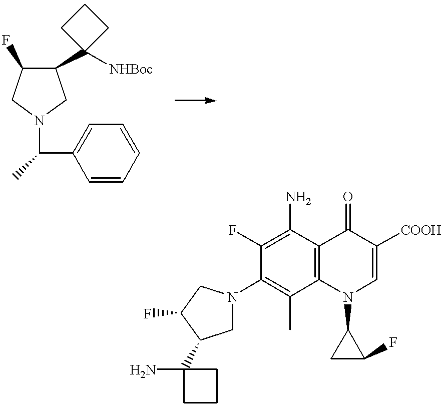

cis 1-[1-(S)-phenylethyl]-4-(1-tert-butoxycarbonylaminocyclobutyl)-3-fluoropyrrolidine (optical isomer B; 1.13 g, 3.12 mmol) was dissolved in ethanol (20 ml). After adding 10% palladium-carbon catalyst (moisture content: 55.6%, 1.0 g), the mixture was stirred under a hydrogen atmosphere at 50° C. for 18 hours. After filtering off the catalyst through celite (washed with methanol), the filtrate was concentrated under reduced pressure. The residue thus obtained was dissolved in dimethyl sulfoxide (10 ml) and 5-amino-6,7-difluoro-1-[2-(S)-fluoro-1-(R)-cyclopropyl]-1,4-dihydro-8-methyl-4-oxoquinoline-3-carboxylic acid (1.18 g, 3.78 mmol) and triethylamine (5 ml) were added thereto. Then the mixture was stirred in a nitrogen atmosphere at 140° C. for 4 days. After allowing to cool, dimethyl sulfoxide was evaporated under reduced pressure and the residue was dissolved in chloroform (50 ml) and washed successively with a 10% aqueous solution of citric acid (50 ml) and a saturated aqueous solution of sodium chloride (100 ml). The organic layer was dried over anhydrous sodium sulfate. After filtering, the filtrate was concentrated under reduced pressure. The residue was then subjected to flash silica gel column chromatography (eluent: chloroform:methanol=9:1) and the eluate was concentrated under reduced pressure. Under ice cooling, conc. hydrochloric acid (5 ml) was dropped into the residue followed by stirring at room temperature for 30 minutes. After adding 1 N hydrochloric acid (30 ml), the liquid reaction mixture was washed with chloroform (50 ml×2) and its pH value was adjusted to 12.0 with an aqueous solution of sodium hydroxide. The liquid reaction mixture was washed with chloroform (100 ml) and then its pH value was adjusted to 7.4 with 1 N hydrochloric acid followed by extraction with chloroform (150 ml×3). The combined organic layer was dried over anhydrous sodium sulfate and filtered. The filtrate was concentrated under reduced pressure and the residue was purified by preparative TLC (developed at the bottom layer of chloroform:methanol:water=7:3:1) to give the title compound as a crude product. After recrystallized from ethanol/ether, 157 mg (17%) of the title compound was obtained.

Melting point: 177-184° C.

¹H—NMR (400 MHz, CDCl₃) δ: 1.16-2.34 (13H, m), 2.47-2.60 (1H, m), 3.35 (1H, t, J=8.79 Hz), 3.53 (1H, q, J=12.21 Hz), 3.78-3.83 (1H, m), 4.09-4.21 (2H, m), 4.76-4.95 (1H, m), 5.42 (1H, dt, J=3.41, 55.18 Hz), 6.53 (2H, brs), 8.60 (1H, d, J=3.41 Hz).

Elemental analysis data: as C₂₂H₂₅F₃N₄O₃•0.5H₂O calcd.: C, 57.51; H, 5.70; N, 12.19 found: C, 57.59; H, 5.52; N, 11.89.

Acute Toxicity

A solution of 5-amino-7-[4-(R)-(1-aminocyclopropyl)-3-(S)-fluoro-1-pyrrolidinyl]-6-fluoro-1-[2-(S)-fluoro-1-(R)-cyclopropyl]-1,4-dihydro-8-methyl-4-oxoquinoline-3-carboxylic acid (abbreviated as cis) hydrochloride (Example 2) or 5-amino-7-[4-(R)-(1-aminocyclopropyl)-3-(R)-fluoro-1-pyrrolidinyl]-6-fluoro-1-(2-(S)-fluoro-1-(R)-cyclopropyl]-1,4-dihydro-8-methyl-4-oxoquinoline-3-carboxylic acid (abbreviated as trans) hydrochloride in distilled water for injection was administered intravenously to Slc:ddy male mice (five mice per group) and the symptoms are observed. The results are as follows.

Results;

| Compound | Dose | Number of death |
|---|---|---|
| cis | 150 mg/kg | 0/5 |
| trans | 150 mg/kg | 2/2* |
| trans | 100 mg/kg | 2/2* |
| trans | 50 mg/kg | 0/5 |

“*”The mouse died immediately after the administration is finished. The test was stopped at the second mouse died.

The same result was obtained for other compounds (Examples 1, 3, 4, 5, 8 and 9) of the present invention at a dose of 150 mg/kg in the test illustrated above.

| Strain/Compound | | | | | |
|---|---|---|---|---|---|
| (Example No.) | 2 | 3 | 5 | 6 | 8 |
| E. coli, NIHJ | ≦0.003 | 0.006 | ≦0.003 | 0.010 | 0.006 |
| S. flexneli, 2A 5503 | ≦0.003 | 0.006 | 0.006 | 0.010 | 0.013 |
| Pr. vulgaris, 08601 | 0.013 | 0.05 | 0.006 | 0.025 | 0.10 |
| Pr. mirabilis, IFO-3849 | 0.025 | 0.10 | 0.05 | 0.10 | 0.10 |
| Ser. marcescens, 10100 | 0.05 | 0.20 | 0.10 | 0.10 | 0.20 |
| Ps. aeruginosa, 32104 | 0.10 | 0.39 | 0.20 | 0.39 | 0.39 |
| Ps. aeruginosa, 32121 | 0.05 | 0.20 | 0.10 | 0.10 | 0.20 |
| X. maltophilia, 11D-1275 | 0.05 | 0.20 | 0.20 | 0.39 | 0.39 |
| S. aureus, 209P | ≦0.003 | ≦0.003 | ≦0.003 | 0.006 | 0.006 |
| S. epidermidis, 56500 | ≦0.003 | 0.006 | 0.013 | 0.025 | 0.013 |
| Str. pyogenes, G-36 | ≦0.003 | 0.013 | 0.006 | 0.010 | 0.025 |
| Str. faecalis, ATCC-19433 | 0.025 | 0.05 | 0.025 | 0.05 | 0.05 |
| S. aureus, 870307 | 0.025 | 0.025 | 0.025 | 0.20 | 0.20 |

| Strain/Compound (Example No.) | 6 | 8 |
|---|---|---|
| E. coli, NIHJ | 0.010 | 0.006 |
| S. flexneli, 2A 5503 | 0.010 | 0.013 |
| Pr. vulgaris, 08601 | 0.025 | 0.10 |
| Pr. mirabilis, IFO-3849 | 0.10 | 0.10 |
| Ser. marcescens, 10100 | 0.10 | 0.20 |
| Ps. aeruginosa, 32104 | 0.39 | 0.39 |
| Ps. aeruginosa, 32121 | 0.10 | 0.20 |
| X. maltophulia, 11D-1275 | 0.39 | 0.39 |
| S. aureus, 209P | 0.006 | 0.006 |
| S. epidermidis, 56500 | 0.025 | 0.013 |
| Str. pyogenes, G-36 | 0.010 | 0.025 |
| Str. faecalis, ATCC-19433 | 0.05 | 0.05 |
| S. aureus, 870307 | 0.20 | 0.20 |

### INDUSTRIAL APPLICABILITY

Because of being excellent in antimicrobial activity and safety, the compounds of the present invention are useful as drugs.

1. A compound represented by formula (I), its salts and hydrates thereof: 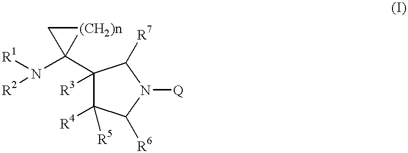
wherein R¹ represents a hydrogen atom or an alkyl group having 1 to 6 carbon atoms;
R² represents a hydrogen atom or an alkyl group having 1 to 6 carbon atoms, provided that said alkyl group may have one or more substituents selected from the group consisting of a hydroxyl group, a halogen atom and an alkoxyl group having 1 to 6 carbon atoms;
R³ and R⁵, each represents a hydrogen atom;
R⁴ represents a hydroxyl group, a halogen atom, a carbamoyl group, an alkyl group having 1 to 6 carbon atoms, an alkoxyl group having 1 to 6 carbon atoms or an alkylthio group having 1 to 6 carbon atoms, provided that said alkyl group may have one or more substituents selected from the group consisting of a hydroxyl group, a halogen atom and an alkoxyl group having 1 to 6 carbon atoms and R⁴ and the substituent on the pyrrolidine ring of the following formula: 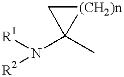
are located in the cis-configuration;
R⁶ and R⁷₁ each independently represents a hydrogen atom or an alkyl group having 1 to 6 carbon atoms;
n is an integer of from 1 to 3; and
Q represents a partial structure represented by the following formula: 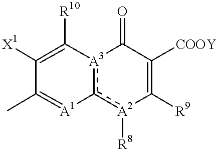
wherein R⁸ represents an alkyl group having 1 to 6 carbon atoms, an alkenyl group having 2 to 6 carbon atoms, a halogenoalkyl group having 1 to 6 carbon atoms, a substituted or unsubstituted cyclic alkyl group having 3 to 6 carbon atoms, a substituted or unsubstituted aryl group, a substituted or unsubstituted heteroaryl group, an alkoxyl group having 1 to 6 carbon atoms or an alkylamino group having 1 to 6 carbon atoms;
R⁹ represents a hydrogen atom or an alkylthio group having 1 to 6 carbon atoms;
R⁹ and R⁸ may form together with a part of the mother nucleus a cyclic structure optionally containing a sulfur atom as a constituting atom thereof and optionally having an alkyl group having 1 to 6 carbon atoms as a substituent;
R¹⁰ represents a hydrogen atom, an amino group, a hydroxyl group, a thiol group, a halogenomethyl group, an alkyl group having 1 to 6 carbon atoms, an alkenyl group having 2 to 6 carbon atoms, an alkynyl group having 2 to 6 carbon atoms or an alkoxyl group having 1 to 6 carbon atoms, provided that said amino group may have one or more substituents selected from the group consisting of a formyl group, an alkyl groups having 1 to 6 carbon atom and an acyl groups having 2 to 5 carbon atoms;
X¹ represents a halogen atom or a hydrogen atom;
A¹ represents a nitrogen atom or a partial structure represented by the following formula (II): 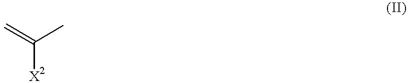
wherein X² represents a hydrogen atom, an amino group, a halogen atom, a cyano group, a halogenomethyl group, a halogenomethoxyl group, an alkyl group having 1 to 6 carbon atoms, an alkenyl group having 2 to 6 carbon atoms, an alkynyl group having 2 to 6 carbon atoms or an alkoxyl group having 1 to 6 carbon atoms, provided that said amino group may have one or more substituents selected from the group consisting of a formyl group, an alkyl group having 1 to 6 carbon atoms and an acyl group having 2 to 5 carbon atoms, and X² and R⁸ may form together with a part of the mother nucleus a cyclic structure optionally containing an oxygen atom, a nitrogen atom or a sulfur atom as a constituting atom thereof and optionally having an alkyl group having 1 to 6 carbon atoms as a substituent;
A² and A³, each represents a nitrogen atom or a carbon atom, provided that A² and A³ may form together with the carbon atom to which they are bonded a partial structure represented by the following formula:
>C═C (A¹═)—N(R⁸)—
or a partial structure represented by the following formula:
>N—C(A¹═)═C(R⁸)—; and
Y represents a hydrogen atom, a phenyl group, an acetoxymethyl group, a pivaloyloxymethyl group, an ethoxycarbonyl group, a choline group, a dimethylaminoethyl group, a 5-indanyl group, a phthalidinyl group, a 5-alkyl-2-oxo-1,3-dioxol-4-ylmethyl group, a 3-acetoxy-2-oxobutyl group, an alkyl group having 1 to 6 carbon atoms, an alkoxymethyl group having 2 to 7 carbon atoms or a phenylalkyl group composed of an alkyl group having 1 to 6 carbon atoms and a phenyl group.

2. The compound as claimed in claim 1, wherein Q in the formula (I) has a structure represented by the following formulae, its salts and hydrates thereof: 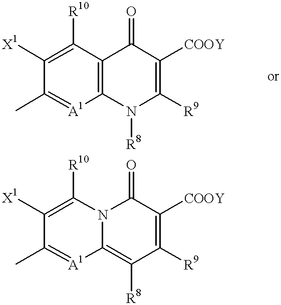
wherein R⁸, R⁹, R¹⁰, A¹, X¹ and Y are each as defined above.

3. The compound as claimed in claim 1 wherein Q in the formula (I) has a structure represented by the following formula, its salts and hydrates thereof: 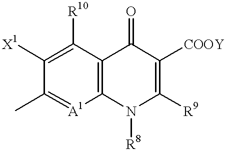
wherein R⁸, R⁹, R¹⁰, A¹, X¹ and Y are each as defined above.

4. The compound as claimed in claim 1, 2 or 3, wherein Q in the formula (I) is a 6-carboxy-9-fluoro-2,3-dihydro-3-(S)-methyl-7-oxo-7H-pyrido[1,2,3-de][1,4]-benzoxazin-10-yl group, its salts and hydrates thereof.

5. The compound as claimed in claim 1, 2 or 3, wherein Q in the formula (I) is an 8-amino-6-carboxy-9-fluoro-2,3-dihydro-3-(S)-methyl-7-oxo-7H-pyrido[1,2,3-de][1,4]benzoxazin-10-yl group, its salts and hydrates thereof.

6. The compounds as claimed in claim 1, 2 or 3 wherein Q in the formula (I) is a 5-amino-3-carboxy-6-fluoro-1-[2-(S)-fluoro-1-(R)-cyclopropyl]-1,4-dihydro-8-methoxy-4-oxoquinolin-7-yl group, its salts and hydrates thereof.

7. The compound as claimed in claim 1, 2 or 3, wherein Q in the formula (I) is a 5-amino-3-carboxy-6-fluoro-1-[2-(S)-fluoro-1-(R)-cyclopropyl]-1,4-dihydro-8-methyl-4-oxoquinolin-7-yl group, its salts and hydrates thereof.

8. The compound as claimed in claim 1, 2 or 3, wherein Q in the formula (I) is a 3-carboxy-6-fluoro-1-[2-(S)-fluoro-1-(R)-cyclopropyl]-1,4-dihydro-8-methoxy-4-oxoquinolin-7-yl group, its salts and hydrates thereof.

9. The compound as claimed in claim 1, 2 or 3, wherein the substituent R⁴ is a halogen atom, its salts and hydrates thereof.

10. The compound as claimed in claim 1, 2 or 3, wherein the substituent R⁴ is a fluorine atom, its salts and hydrates thereof.

11. The compound as claimed in claim 1, 2 or 3, wherein n is 1 or 2, its salts and hydrates thereof.

12. The compound as claimed in claim 1, 2 or 3, wherein n is 1, its salts and hydrates thereof.

13. The compound as claimed in claim 1, 2 or 3, wherein the substituent R⁴ is a fluorine atom and n is 1 or 2, its salts and hydrates thereof.

14. The compound as claimed in claim 1, 2 or 3, wherein the substituent R⁴ is a fluorine atom and n is 1, its salts and hydrates thereof.

15. The compound as claimed in claim 1, 2 or 3, wherein the substituent R⁸ is a halogenocyclopropyl group, its salts and hydrates thereof.

16. The compound as claimed in claim 1, 2 or 3, wherein the substituent R⁸ is a 1,2-cis-2-halogenocyclopropyl group, its salts and hydrates thereof.

17. The compound as claimed in claim 1, 2 or 3, wherein the substituent R⁸ is a stereochemically pure one, its salts and hydrates thereof.

18. The compound as claimed in claim 1, 2 or 3, wherein the substituent R⁸ is a (1R,2S)-2-halogenocyclopropyl group, its salts and hydrates thereof.

19. The compound as claimed in claim 1, 2 or 3, wherein the substituent R⁸ is a (1R,2S)-2-fluorocyclopropyl group, its salts and hydrates thereof.

20. The compound as claimed in claim 1, 2 or 3, wherein the substituent X¹ is a halogen atom, its salts and hydrates thereof.

21. The compound as claimed in claim 20, wherein said halogen atom is a fluorine atom, its salts and hydrates thereof.

22. The compound represented by the formula (I) as claimed in claim 1, 2 or 3, which is stereochemically pure one, its salts and hydrates thereof.

23. 10-[4-(R)-(1-Aminocyclopropyl)-3-(S)-fluoro-1-pyrrolidinyl]-9-fluoro-2,3-dihydro-3-(S)-methyl-7-oxo-7H-pyrido[1,2,3-de][1,4]benzoxazine-6-carboxylic acid, its salts and hydrates thereof.

24. 8-Amino-10-[4-(R)-(1-aminocyclopropyl)-3-(S)-fluoro-1-pyrrolidinyl]-9-fluoro-2,3-dihydro-3-(S)-methyl-7-oxo-7H-pyrido[1,2,3-de][1,4]benzoxazine-6-carboxylic acid, its salts and hydrates thereof.

25. 5-Amino-7-[4-(R)-(1-aminocyclopropyl)-3-(S)-fluoro-1-pyrrolidinyl]-6-fluoro-1-[2-(S)-fluoro-1-(R)-cyclopropyl]-1,4-dihydro-8-methyl-4-oxoquinoline-3-carboxylic acid, its salts and hydrates thereof.

26. 5-Amino-7-[4-(R)-(1-aminocyclopropyl)-3-(S)-fluoro-1-pyrrolidinyl]-6-fluoro-1-[2-(S)-fluoro-1-(R)-cyclopropyl]-1,4-dihydro-8-methoxy-4-oxoquinoline-3-carboxylic acid, its salts and hydrates thereof.

27. 7-[4-(R)-(1-Aminocyclopropyl)-3-(S)-fluoro-1-pyrrolidinyl]-6-fluoro-1-[2-(S)-fluoro-1-(R)-cyclopropyl]-1,4-dihydro-8-methoxy-4-oxoquinoline-3-carboxylic acid, its salts and hydrates thereof.

28. A drug containing as the active ingredient the compound as claimed in claim 1, 2 or 3, hydrates thereof, salts of said compound or hydrates of said salts.

29. An antimicrobial agent containing as the active ingredient the compound as claimed in claim 1, 2 or 3, hydrates thereof, salts of said compound or hydrates of said salts.

30. A compound represented by the following formula, its salts and hydrates thereof: 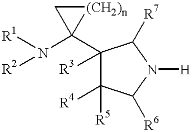
wherein R¹ represents a hydrogen atom or an alkyl group having 1 to 6 carbon atoms;
R² represents a hydrogen atom or an alkyl group having 1 to 6 carbon atoms, provided that said alkyl group may have one or more substituents selected from the group consisting of a hydroxyl group, a halogen atom and an alkoxyl group having 1 to 6 carbon atoms;
R³ and R⁵, each represents a hydrogen atom;
R⁴ represents a hydroxyl group, a halogen atom, a carbamoyl group, an alkyl group having 1 to 6 carbon atoms, an alkoxyl group having 1 to 6 carbon atoms or an alkylthio group having 1 to 6 carbon atoms, provided that said alkyl group may have one or more substituents selected from the group consisting of a hydroxyl group, a halogen atom and an alkoxyl group having 1 to 6 carbon atoms, and R⁴ and the substituent on the pyrrolidine ring of the following formula: 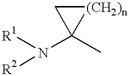
are located in the cis-configuration;
R⁶ and R⁷, each independently represents a hydrogen atom or an alkyl group having 1 to 6 carbon atoms; and
n is an integer of from 1 to 3.

31. The compound as claimed in claim 30, wherein the substituent R⁴ is a halogen atom, its salts and hydrates thereof.

32. The compound as claimed in claim 30, wherein the substituent R⁴ is a fluorine atom, its salts and hydrates thereof.

33. The compound as claimed in claim 30, 31 or 32, wherein n is 1 or 2, its salts and hydrates thereof.

34. The compound as claimed in claim 30, 31 or 32, wherein n is 1, its salts and hydrates thereof.

35. The compound as claimed in claim 30, wherein the substituent R⁴ is a fluorine atom and n is 1 or 2, its salts and hydrates thereof.

36. The compound as claimed in claim 30, wherein the substituent R⁴ is a fluorine atom and n is 1, its salts and hudrates thereof.

37. 4-(R)(1-Aminocyclopropyl)-3-(S)-fluoro-1-pyrrolidine, its salts and hydrates thereof.
